(19) United States
(12) Patent Application Publication
(10) Pub. No.: US 2025/0064354 A1
(43) Pub. Date: Feb. 27, 2025

(54) MULTIMODAL CONTINUOUS MONITORING DEVICE

(71) Applicant: Kilele Health, Oakwood, OH (US)

(72) Inventor: Jason Heikenfeld, Cincinnati, OH (US)

(21) Appl. No.: 18/815,439

(22) Filed: Aug. 26, 2024

Related U.S. Application Data
(60) Provisional application No. 63/578,705, filed on Aug. 25, 2023.

(51) Int. Cl. A61B 5/145; A61B 5/00; A61B 5/0205; A61B 5/024; A61B 5/1473; A61B 5/266

(52) U.S. Cl. CPC A61B 5/14542; A61B 5/0205; A61B 5/1473; A61B 5/266; A61B 5/6833; A61B 5/024

(57) ABSTRACT

A wearable heart monitoring device includes a carrier and a housing. The carrier includes a first electrode defining a sensor configured to measure at least one of b-type natriuretic peptide (BNP), N-terminal pro b-type natriuretic peptide (NT-proBNP), or Troponin. Moreover, a second electrode is configured to measure at least one of parameter associated with a heart, heart disease, or heart disease treatment, of a patient wearing the heart monitoring device. The housing includes an adhesive thereon. The housing is coupled to the carrier. Moreover, the housing contains a controller communicably coupled to the first electrode and the second electrode, memory coupled to the controller, and a communication interface communicably coupled to the controller.

### CROSS-REFERENCE TO RELATED APPLICATIONS

This application claims the benefit of U.S. Provisional Patent Application Ser. No. 63/578,705, filed Aug. 25, 2023, having the title “MULTIMODAL CONTINUOUS MONITORING DEVICE”, the disclosure of which is hereby incorporated herein by reference.

### FIELD OF THE INVENTION

Aspects herein relate generally to multimodal continuous monitoring devices. Further, aspects herein relate to aptamer sensors with additional sensing capabilities not typically measured with aptamers alone. Yet further, aspects herein relate to wearable monitoring devices, such as a wearable heart monitoring device.

### DESCRIPTION OF THE RELATED ART

Wearable biochemical sensors have a powerful potential for personalized medicine and continuous health monitoring. However, biochemical sensors, e.g., that chemically measure an analyte in-vivo, are typically limited to single-sensor modalities such as those that exist for continuous glucose monitors.

### BRIEF SUMMARY

According to aspects herein, a sensing device is provided for sensing at least one analyte in a fluid (e.g., a fluid such as dissolved oxygen). For instance, a first sensor can comprise at least one analyte detecting material adjacent to an associated (first) electrode. As an example, the first sensor may be implemented as an electrochemical aptamer sensor comprising one or more aptamers. In addition, at least one oxygen limiting material can be provided adjacent to an electrode, which may be the same as, or different from, the associated electrode of the first sensor. For instance, in a first example configuration, the oxygen limiting material is adjacent to the first electrode. In another example configuration, the analyte detecting material is adjacent to the first electrode, etc.

As a further example, the first sensor can integrate an aptamer sensing capability that also includes at least one oxygen limiting material. In an example implementation, the oxygen limiting material is a monolayer blocking layer. In yet another example implementation, the oxygen limiting material is non-monolayer blocking layer. In yet a further example, the oxygen limiting material is silicon dioxide. Regardless, the oxygen limiting material may further comprise an antifouling chemistry and/or a plurality of aptamers.

Moreover, the aptamer can comprise a redox tag that is electrochemically measured by electron-transfer through the oxygen limiting material. Dissolved oxygen can be measured, for instance, by diffusion limited transport of oxygen through the oxygen limiting material and subsequent oxygen reduction current.

As noted herein, the sensing device can comprise multiple electrodes (e.g., each associated with potentially, a different sensor). Each electrode can be incorporated in the same measurement circuit. Thus, multiple measurements can be performed in a single measurement scan. The measurement scan can be implemented, for instance, by square wave voltammetry.

In still another example configuration, the sensing device comprises at least working and counter electrodes. Oxygen limiting material can be placed on the counter electrode and an analyte detecting material can be placed on the working electrode. In another configuration, the oxygen limiting material is placed on the working electrode and/or the oxygen limiting material is located between an analyte detecting material and the working electrode.

Regardless, the analyte detecting material may comprise an enzyme for detection of glucose. As a further example, the analyte may comprise potassium. As yet a further example, the analyte may comprise b-type natriuretic peptide (BNP), N-terminal pro b-type natriuretic peptide (NT-proBNP) or Troponin.

In still another example, a continuous sensing device is provided for sensing potassium. For instance, at least one electrode can comprise a plurality of potassium binding aptamers and a blocking layer selected from the group consisting of a monolayer blocking layer and non-monolayer blocking layer. In addition, a means can be provided for measuring potassium over time. Further, a means can be provided for calculating a score of percentage time in range for potassium. As an example, the means for calculating a score of percentage time in range for potassium can be capable of providing the score on an ongoing basis for a period of time selected from the group consisting of hourly, semi-daily, daily, multiday, weekly, biweekly, monthly, and yearly.

According to yet further aspects herein, a wearable heart monitoring device is provided. The wearable heart monitoring device comprises a first electrode defining a sensor configured to measure at least one of b-type natriuretic peptide (BNP), N-terminal pro b-type natriuretic peptide (NT-proBNP) or Troponin. A second electrode is configured to measure at least one parameter associated with heart disease or heart disease treatment of a patient wearing the heart monitoring device. For instance, the second electrode can be configured to measure at least one of potassium, creatinine or Troponin. Additionally, a housing is coupled to the first electrode and the second electrode. The housing contains at least a potentiostat coupled to at least the first electrode, a controller communicably coupled to the potentiostat, memory coupled to the controller, and a communication interface communicably coupled to the controller.

In some implementations of the wearable heart monitoring device, the first electrode is configured to measure at least one of b-type natriuretic peptide (BNP) or N-terminal pro b-type natriuretic peptide (NT-proBNP) and the second electrode is configured to measure at least one of potassium, creatinine or Troponin. In an alternative configuration, the second electrode can measure potassium and a third sensor can be provided, which is configured to measure creatinine.

In some implementations of the wearable heart monitoring device, the first or second electrode is configured to measure oxygen concentration.

As an additional example, in some implementations, the first electrode or the second electrode functions as a standby sensor for detecting a heart attack by measuring Troponin.

As yet a further example, some implementations of the wearable heart monitoring device further comprise a gel electrode. With respect to the first electrode and/or the second electrode, the gel electrode is utilized for heart rate monitoring, and functions as a counter and/or reference for the first electrode and/or the second electrode.

Moreover, the first electrode and the second electrode can be provided on a carrier, where the carrier projects outwardly from the housing such that when applied to the patient, the gel pad electrode of the housing is operatively configured to be adherable outside the body, whereas the first electrode and the second electrode puncture the skin so as to reside inside the body.

Some implementations of the wearable heart monitoring device further comprise at least one additional sensor. For instance, an additional sensor can be selected from an optical heart rate sensor, an optical oxygen saturation sensor, an accelerometer, an electrocardiogramansor, an optical pulse wave sensor configured for measuring blood pressure, or an RF-measured thoracic-fluid index sensor.

Moreover, an example implementation of the wearable heart monitoring device comprises at least three sensors for simultaneous monitoring by the controller, of breathing rate, blood oxygen saturation (SpO2), and tissue oxygenation, so as to provide a measure of pulmonary-cardio-vascular performance.

In some implementations of the wearable heart monitoring device, the first electrode is initially not used. Here, the second electrode is configured as an electrocardiogram. Moreover, the controller monitors the output of the second electrode (electrocardiogram) and issues an alert to have the first electrode inserted into the patient for monitoring thereof, if the monitored output of the second electrode (electrocardiogram) meets a predetermined criterion.

In some implementations of the wearable heart monitoring device, a second sensor can alert the controller that a first sensor should be inserted into the patient. Insertion of the first sensor can be performed using any suitable technique, even a separate device co-located on skin. By way of a non-limiting but illustrative example, the first electrode is invasive and is initially not used. The second electrode is non-invasive, and is configured as an electrocardiogram. Here, the controller monitors the output of the second electrode (electrocardiogram) and issues an alert to have the first electrode installed if the monitored output meets a predetermined criterion. For instance, the first electrode can be inserted through an aperture in the housing, or other techniques can be applied.

In yet other implementations of the wearable heart monitoring device, the controller is operatively configured to collect heart analytical data based upon measurements collected from the first electrode and the second electrode, and store the results in memory. Moreover, the controller is further operatively configured to extract the heart analytical data from the memory and convey the data to a remote processing device.

According to yet further aspects herein, a wearable heart monitoring device is provided. The wearable heart monitoring device comprises a housing having an adhesive thereon, where the adhesive is provided for securing the housing to a patient wearing the heart monitoring device. A first electrode is configured to measure a first cardiovascular parameter of a patient wearing the heart monitoring device. Likewise, a second electrode is configured to measure a second cardiovascular parameter of the patient wearing the heart monitoring device. Also, a third electrode functions as a counter electrode for at least one of the first electrode and the second electrode. Under this configuration, the first electrode and the second electrode are operationally configured as at least one needle electrode that is capable of puncturing through an epidermis of the patient, and the third electrode is operationally configured to attach to the outer skin of the patient. Moreover, the housing contains at least a potentiostat, a controller, memory, and a communication interface. The potentiostat couples to at least the first electrode. Moreover, the controller is communicably coupled to the potentiostat, the memory is coupled to the controller, and the communication interface is communicably coupled to the controller.

In some implementations of the wearable heart monitoring device, the first electrode can be configured to measure the first cardiovascular parameter of the patient by measuring at least one of b-type natriuretic peptide (BNP) or N-terminal pro b-type natriuretic peptide (NT-proBNP). The second electrode can be configured to measure the second cardiovascular parameter of the patient by measuring at least one of potassium, creatinine or troponin.

The first electrode or the second electrode can be configured to measure oxygen concentration.

In certain implementations, the third electrode can measure at least one of an electrocardiogrameasurement, heart rate (HR), heart rate variability (HRV), HR+HRV, Oxygen desaturation, blood pressure, breathing rate, or combinations thereof.

In some other implementations of the wearable heart monitoring device, the controller can adjust a voltage scanning window, thus eliminating the need for a reference electrode.

According to yet further aspects herein, a wearable heart monitoring device is provided. The wearable heart monitoring device comprises a housing for securing to a patient wearing the heart monitoring device. The wearable heart monitoring device also comprises a first electrode configured to measure a first cardiovascular parameter of a patient wearing the heart monitoring device, and a second electrode configured to measure a second cardiovascular parameter of the patient wearing the heart monitoring device. In use, the first electrode and the second electrode puncture through an epidermis of the patient. Moreover, the housing contains at least a potentiostat that couples to at least the first electrode, a controller communicably coupled to the potentiostat, memory coupled to the controller, and a communication interface communicably coupled to the controller.

In some implementations of the wearable heart monitoring device, the first electrode is configured to measure the first cardiovascular parameter of the patient by measuring at least one of b-type natriuretic peptide (BNP) or N-terminal pro b-type natriuretic peptide (NT-proBNP), and the second electrode is configured to measure the second cardiovascular parameter of the patient by measuring at least one of potassium, creatinine or Troponin.

Yet further, in some embodiments of the wearable heart monitoring device, the controller adjusts a voltage scanning window thus eliminating the need for a reference electrode.

According to still further aspects herein, a wearable monitoring device comprises a first electrode and a housing. The first electrode measures at least one target analyte and at least one oxygen characteristic. The housing is coupled to the first electrode, and contains at least a potentiostat communicably coupled to the first electrode, a controller communicably coupled to the potentiostat, memory communicably coupled to the controller, and a communication interface communicably coupled to the controller. In use, the potentiostat collects a measurement from the first electrode, the controller stores the measurement from the potentiostat in memory, and the measurement is wirelessly communicated via the communication interface.

For instance, the first electrode can comprise an aptamer sensor further configured for measuring the at least one oxygen characteristic. As another example, the first electrode can comprise an enzymatic sensor having an oxygen limiting layer on the electrode such that a single electrode measures the at least one target analyte and the at least one oxygen characteristic. Yet further, the wearable monitoring device can comprise at least one additional electrode comprising a select one of an optical sensor, a strain sensor, an electrical sensor, and an acceleration sensor.

According to additional aspects herein, a wearable monitoring device comprises a first electrode that measures at least one target analyte, a counter electrode biased to measure oxygen reduction, and a housing coupled to the first electrode. The housing contains at least a potentiostat coupled to the first electrode, a controller communicably coupled to the potentiostat, memory coupled to the controller, and a communication interface communicably coupled to the controller. In use, the potentiostat collects a measurement from the first electrode, the controller stores the measurement from the potentiostat in memory, and the controller wirelessly communicates the measurement via the communication interface.

In some implementations, the wearable monitoring device can further comprise an antifouling chemistry on the oxygen limiting material so as to preserve measurement accuracy in-vivo. In some other implementations, the wearable monitoring device further comprises at least one additional electrode comprising a select one of an optical sensor, a strain sensor, an electrical sensor, and an acceleration sensor.

According to still further aspects herein, a wearable monitoring device comprises a first sensor implementing non-invasive sensor, and a housing having an aperture therethrough. The housing contains at least a potentiostat communicably coupled to the first sensor, a controller communicably coupled to the potentiostat, memory communicably coupled to the controller, and a communication interface communicably coupled to the controller. In use, the potentiostat collects a measurement from the first sensor, the controller stores the measurement from the potentiostat in memory, and wirelessly communicates the measurement via the communication interface. Moreover, the controller detects for at least one condition based upon the measurements from the first sensor, and upon the controller detecting the at least one condition, the controller provides a signal to trigger a second sensor to be added to the monitoring device. The second sensor defines an invasive sensor that is inserted through the aperture of the housing, where the second sensor measures an analyte.

In some implementations, the first sensor can comprise an electrocardiogra sensor. Here, the at least one condition comprises a heart condition of a patient wearing the monitoring device, and the second sensor measures an analyte comprising at least one of NT-proBNP or Troponin.

### BRIEF DESCRIPTION OF THE SEVERAL VIEWS OF THE DRAWINGS

FIG. 1 is a schematic illustration of a wearable monitoring device using aptamers and a monolayer oxygen diffusion limiting layer, which also provides antifouling capability.

FIG. 2 is a schematic illustration of a wearable monitoring device using aptamers and a non-monolayer oxygen diffusion limiting layer.

FIG. 3 is a schematic illustration of a wearable monitoring device using aptamers and a non-monolayer oxygen diffusion limiting layer with addition of an anti-fouling layer.

FIG. 4 is a graph showing an example sensor response vs. analyte concentration demonstration of an example device, e.g., implemented according to FIG. 2 or FIG. 3, for the sensing of phenylalanine.

FIG. 5A is a graph showing an example plot of voltage vs Redox Tag Current.

FIG. 5B is a graph showing a square wave voltammogram demonstration of an example device, e.g., implemented according to FIG. 2 or FIG. 3, for the sensing of phenylalanine and of dissolved oxygen concentration.

FIG. 6 is a schematic illustration of an example wearable monitoring device for multimodal sensing.

FIG. 7 is a schematic illustration of an example wearable monitoring device for multimodal sensing where the electrochemical sensor beneath the skin is applied to the device only as needed.

FIG. 8 is an example environment for connecting a wearable monitoring device to one or more additional processing devices.

FIG. 9 is a system level and component level diagram of an example multimodal wearable monitoring device.

FIG. 10 is an example data display from a wearable multimodal monitoring device.

### DETAILED DESCRIPTION

### Definitions

As used herein, a “continuous sensor” means a sensor that changes in response to a changing measure of a biomarker, such as heart rate or the concentration of at least one solute in a solution such as an analyte.

Likewise, “continuous sensing” means sensing, detecting, measuring, or otherwise identifying changes in response to a changing measure of a biomarker, such as heart rate or the concentration of at least one solute in a solution such as an analyte.

Similarly, as used herein, “continuous monitoring” means the capability of a device to provide multiple measurements over time.

In this regard, “continuous” as used above, does not explicitly require sensing without interruption. Rather, “continuous” can include discrete events (sensing, monitoring, etc.) taken over time, where the discrete events are sufficient to capture changes in response to a changing measure of a biomarker, the concentration of at least one solute in a solution such as an analyte, etc.

As used herein, the term “about,” when referring to a value or to an amount of mass, weight, time, volume, pH, size, concentration or percentage is meant to encompass variations of +20% in some implementations, +10% in some implementations, +5% in some implementations, +1% in some implementations, +0.5% in some implementations, and +0.1% in some implementations from the specified amount, as such variations are appropriate to perform the disclosed method.

As used herein, the term “electrode” means any material that is electrically conductive such as gold, platinum, nickel, silicon, conductive liquid infused materials such as ionic liquids, Poly(3,4-ethylenedioxythiophene) polystyrene sulfonate (PEDOT: PSS), conductive oxides, carbon, boron-doped diamond, nanotubes or nanowire meshes, or other suitable electrically conducting materials.

As used herein, the term “blocking layer” means a homogeneous or heterogeneous layer of material or one or more types of molecules on an electrode that reduce electrochemical background current and/or current due to electrochemical interference, and which may promote proper freedom of movement for the aptamer, which is required for creating a measurable response to analyte concentration.

As used herein, the term “non-monolayer blocking layer” means a homogeneous or heterogeneous layer of material or one or more types of molecules on an electrode which do not represent a monolayer configuration, and which reduces electrochemical background current and/or current due to electrochemical interference, and which may promote proper freedom of movement for the aptamer, which is required for creating a measurable response to analyte concentration. For example, a metal or semiconductor oxide can be a non-monolayer blocking layer, or a thin polymer film may be a non-monolayer blocking layer, because they are comprised of multiple layers of atoms or molecules. A single atomic monolayer of SiO₂ for example would be a monolayer, whereas 3 nm of SiO₂ is a non-monolayer.

As used herein, the term “antifouling layer” means a homogeneous or heterogeneous layer of material or of one or more types of molecules on a surface which reduces fouling on a surface compared to if such an antifouling layer was not utilized.

As used herein, the term “oxygen limiting layer” means layer of material placed between an analyte detecting material and an electrode, that limits the diffusion oxygen to the electrode, but allows enough oxygen diffusion such that oxygen is electrochemically measurable, such that the electrode may be used as a sensor of dissolved oxygen, and the oxygen limiting layer permits redox electron transfer such that the analyte can be measured by the analyte detecting material.

As used herein, the term “analyte detecting material” means a layer of material such as aptamers or enzymes or other suitable material that allows for continuous sensing of an analyte.

As used herein, the term “aptamer” means a molecule that undergoes a conformation or binding change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers and other affinity-based probes. Modifications may include substituting unnatural nucleic acid bases for natural bases within the aptamer sequence, replacing natural sequences with unnatural sequences, or other suitable modifications that improve sensor function, but which behave analogous to traditional aptamers. Two or more aptamers bound together can also be referred to as an aptamer (i.e., not separated in solution). In some example implementations, aptamers can have molecular weights of at least 1 kDa, at least 10 kDa, or at least 100 kDa. Aptamers can be electrochemical in nature and tagged with a redox tag such as methylene blue. Aptamers can also be optical in nature such as fluorescent molecular beacon aptamers carried on a waveguide, or fluorescent FRET aptamers with donor and acceptor fluorophores carried on a waveguide, or for example aptamers at the end of a biolayer interferometry probe.

As used herein, the term “optical sensor” means any biosensor that is used for optical biosensing such as heart rate, oxygenation, optical glucose or other suitable measurements.

As used herein, the term “strain sensor” means any sensor that is used for measuring strain, such as by measuring compression and/or stretching, of a surface. An example of a strain sensor is a biosensor such as a strain sensor used for measuring breathing rate.

As used herein, the term “electrical sensor” means any sensor that is used for electrical measurements on the body such as galvanic skin response, electrocardiograph (ECG) or EKG for heart monitoring, or other suitable measurements.

As used herein, the term “acceleration sensor” means any sensor that is for measuring body motion, vibration, or displacement and movement.

As used herein, the term “redox tag” or “redox molecule” means any species such as small or large molecules with a redox active portion that when brought adjacent to an electrode, can reversibly transfer at least one electron with the electrode. Redox tag or molecule examples include methylene blue, ferrocene, quinones, or other suitable species that satisfy the definition of a redox tag or molecule. In some cases, a redox tag or molecule is referred to as a “redox mediator”. Redox tags or molecules may also exchange electrons or change in behavior when brought into proximity with other redox tags or molecules. Exogenous redox molecules are those added to a device, e.g., they are not endogenous and provided by the sample fluid to be tested.

As used herein, the term “change in electron transfer” means a redox molecule whose electron transfer with an electrode has changed in a measurable manner. This change in electron transfer can, for example, originate from an availability for electron transfer, distance from an electrode, diffusion rate to or from an electrode, a shift or increase or decrease in electrochemical activity of the redox molecule, or any other implementation as taught herein that results in a measurable change in electron transfer between the redox molecule and the electrode.

As used herein, the term “sensing monolayer” includes at least a plurality of aptamers on a working electrode, which may also include a plurality of molecules or mixtures of molecules that form a blocking layer or an anti-fouling layer.

As used herein, the term “analyte” means any solute in a biofluid which can be measured using a sensor. Analytes can be small molecules, proteins, peptides, electrolytes, acids, bases, antibodies, molecules with small molecules bound to them, DNA, RNA, drugs, chemicals, pollutants, or other solutes in a biofluid.

### Introduction

Aspects herein relate to multi-modal wearable sensors that facilitate decentralized medical care, and can provide more timely/actionable information for doctors and patients. Unlike glucose in diabetes, in complicated conditions such as cardiovascular disease, multiple measures are often required, and having a patient use or wear multiple diagnostic devices is not practical. Unfortunately, multimodal sensing devices for chronic disease management have been lacking. As such, novel approaches are provided herein, which simplify the integration of biochemical sensing with additional sensing modalities that enhance patient care, health and wellness monitoring, etc.

Throughout, certain ancillary aspects may be described with brevity in order to focus on inventive aspects. As a few examples, certain sensors may be described as simple individual elements for clarity of discussion (e.g., when illustrating desired characteristics and/or functionality). Further, certain sensors may utilize two or more electrodes, reference electrodes, or may otherwise require additional supporting technology or features. Moreover, in practice, sensors can be in duplicate, triplicate, or more, to provide improved data and readings. Also, sensors may provide continuous data and/or readings (e.g., continuous can be implemented by analog, real-time, continuous sensing and/or reading, discrete sensing and/or reading, or combinations thereof). Yet further, certain disclosure herein may describe or illustrate one or more sub-components of a sensing device, where additional sub-components (e.g., a reference or counter electrode, a battery, antenna, adhesive) may be required for use of the device in select applications. Such ancillary aspects may be described in prose, schematically, or in greater detail across two or more FIGURES. As such, the specification should be considered as a whole, allowing for aspects from the FIGURES in any combination deemed practical.

Certain features, capabilities, or processes may be described in terms of ranges. As used throughout, all ranges of parameters disclosed herein include the endpoints of the ranges.

### Electrochemical Aptamer Sensor Example 1

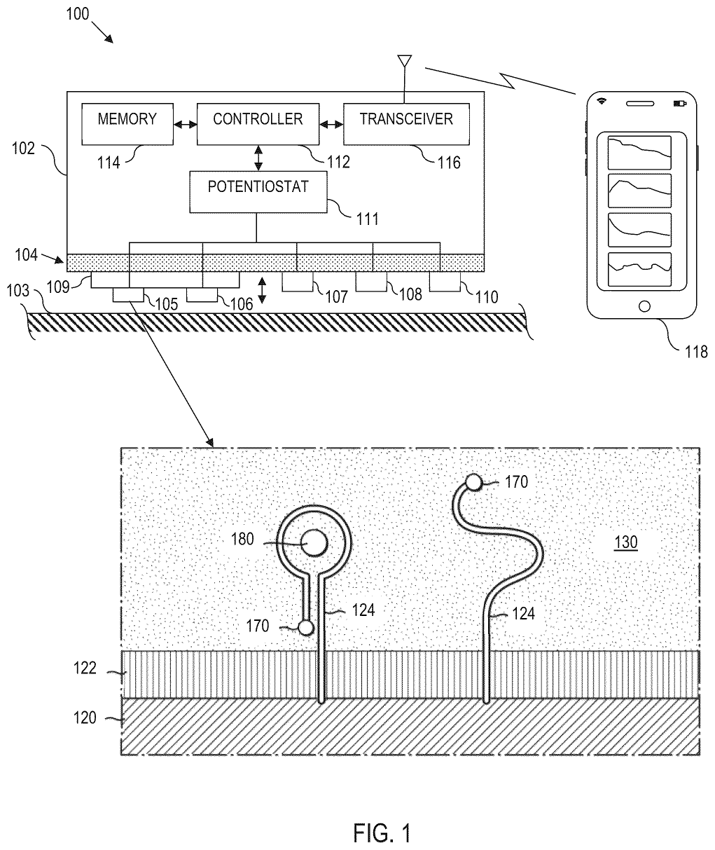

With reference to FIG. 1, a wearable monitoring device is schematically illustrated. In this illustrative example, the wearable monitoring device is implemented as a wearable heart monitoring device 100. However, as will be described more fully herein, the wearable monitoring device can be used for other functions. The wearable heart monitoring device 100 includes a housing 102 that is intended to attach to the skin 103 of a patient. For instance, the housing 102 can optionally include an adhesive 104 thereon, where the adhesive 104 is provided for securing the housing 102 to a patient wearing the wearable heart monitoring device 100. Moreover, alternative means, e.g., a strap, can be provided to attach the housing 102 to the patient.

The wearable heart monitoring device 100 includes in general, a first electrode 105 configured to measure a first cardiovascular parameter of a patient wearing the heart monitoring device, and a second electrode 106 configured to measure a second cardiovascular parameter of the patient wearing the heart monitoring device.

In some example implementations, the first electrode 105 defines a sensor configured to measure at least one of b-type natriuretic peptide (BNP), N-terminal pro b-type natriuretic peptide (NT-proBNP), Troponin, oxygen concentration, etc., as will be described in greater detail herein.

In some example implementations, the second electrode 106 defines as sensor configured to measure at least one parameter associated with heart disease or heart disease treatment of a patient wearing the heart monitoring device. For instance, the second electrode can be configured to measure at least one of potassium, creatinine, Troponin, oxygen concentration, etc.

An optional third electrode 107 is also illustrated. Where provided, the third electrode can measure a parameter, e.g., creatinine. As another example, the third electrode 107 can optionally measure at least one of an electrocardiogrameasurement, heart rate (HR), heart rate variability (HRV), HR+HRV, Oxygen desaturation, blood pressure, breathing rate, or combinations thereof. As yet another example, the third electrode 107 can function as a counter electrode for at least one of the first electrode 105 and the second electrode 106. When functioning as a counter electrode, the third electrode 107 can be operationally configured to attach to the outer skin of the patient.

In some implementations, the wearable heart monitoring device 100 can include a gel pad electrode 108. Here, with respect to the first electrode 105 and/or the second electrode 106, the gel pad electrode 108 is utilized for heart rate monitoring, and functions as a counter and/or reference for the first electrode 105 and/or the second electrode 106.

For instance, in some implementations, the first electrode 105 and the second electrode 106 puncture through an epidermis of the patient. By way of example, the first electrode 105 and the second electrode 106 can be operationally configured as at least one needle electrode that is capable of puncturing through the epidermis. As another example, the first electrode 105 and the second electrode 106 can be provided on a carrier 109 that projects outwardly from the housing 102 such that when applied to the patient, the gel pad electrode 108 is operatively configured to be adherable outside the body, whereas the first electrode 105 and the second electrode 106 puncture the skin so as to reside inside the body.

In some implementations, the wearable heart monitoring device 100 can optionally include at least one additional sensor 110. As a few illustrative examples, an additional sensor 110 can be selected from an optical heart rate sensor, an optical oxygen saturation sensor, an accelerometer, an electrocardiograph (ECG) sensor, an optical pulse wave sensor configured for measuring blood pressure, or an RF-measured thoracic-fluid index sensor. In another example implementation, the at least one additional sensor 110 can include at least three sensors for simultaneous monitoring of breathing rate, blood oxygen saturation (SpO₂), and tissue oxygenation so as to provide a measure of pulmonary-cardio-vascular performance.

In an example implementation, the first electrode 105 or the second electrode 106 functions as a standby sensor for detecting a heart attack by measuring Troponin.

Moreover, in some implementations, the housing 102 contains at least a potentiostat 111 that is coupled to at least one of the included electrodes, e.g., at least the first electrode 105. The housing 102 can also contain a controller 112 communicably coupled to the potentiostat 111, memory 114 coupled to the controller 112, and a communication interface 116 communicably coupled to the controller 112. In some implementations, the controller 112 adjusts a voltage scanning window thus eliminating the need for a reference electrode. Here, the controller 112 analyzes measurements collected from the potentiostat 111, and stores the measurements in the memory 114. The controller also monitors the values of the measurements and can issue alerts if the patient is at risk for an event such as a heart attack.

In some implementations, the communication interface 116, e.g., a Bluetooth, Wi-Fi, cellular, Zigbee, ultrawide-band, or combination thereof, communicates with a remote processing device 118, such as a smartphone running an app to display results collected and processed by the wearable heart monitoring device 100, as will be described in greater detail herein.

By way of illustration, and not by way of limitation, in an example use, the first electrode 105 is initially not used. Here, another sensor (e.g., a select one of the second electrode 106, gel pad electrode 108, or additional sensor 110) is configured as an electrocardiogram. The controller 112 monitors the output of the electrocardiogram and issues an alert to have the first electrode 105 inserted into the patient for monitoring thereof, if the monitored output of the electrocardiogram meets a predetermined criterion.

In another example embodiment of the wearable heart monitoring device 100, the controller 112 is operatively configured to collect heart analytical data based upon measurements collected from the first electrode 105 and the second electrode 106, and store the results in the memory 114. Additionally, the controller 112 is further operatively configured to extract the heart analytical data from the memory 114 and convey the data to a remote processing device 118, e.g., a smartphone, remote server, combinations thereof, etc.

### Operation of an Example Electrode

By way of illustrative example, an exploded view schematically illustrates aspects of a sensor, such as the sensor associated with one or more of the above-described electrodes, e.g., the first electrode 105. As illustrated, the sensor includes at least one working electrode 120, at least one blocking layer 122, and at least one aptamer 124. The sensor is placed initially in a sample fluid 130, such as subcutaneous interstitial fluid, as shown. In this placement, a select blocking layer 122 is positioned between the sample fluid 130 and a corresponding working electrode 120.

Each working electrode 120 may comprise a suitable electrode material, such as gold, carbon, or other suitable electrode material, as described more fully herein. Each blocking layer 122 (e.g., a monolayer blocking layer), may include a plurality of molecules (such as mercaptooctanol or other suitable molecules that are thiol bonded to the corresponding working electrode 120).

In the illustrated implementation, each aptamer 124 contacts an associated working electrode 120, couples to or otherwise through a corresponding blocking layer 122, and extends into the sample fluid 130.

A redox tag 170 (e.g., methylene blue) is associated with at least one aptamer 124, such as by being bound thereto. For instance, in FIG. 1, each aptamer 124 includes a redox tag 170 at a distal end thereof, where the distal end is situated in the sample fluid 130. Moreover, as illustrated each aptamer 124 is responsive to binding to an analyte 180. For purposes of illustration, the left-most aptamer 124 in FIG. 1 is shown bound to an analyte 180, whereas the right-most aptamer 124 of FIG. 1 is shown capable of binding to an analyte 180.

In the illustrative example taught for FIG. 1, the aptamer 124 is a simple stem loop (hairpin) aptamer where analyte 180 binding causes the stem loop to form, and a redox current measured from the redox tag 170 to increase. Here, the redox current can be measured, e.g., via the potentiostat 111, using square wave voltammetry, chronoamperometry, or other suitable technique. In the absence of analyte 180 binding to the aptamer 124, the stem loop conformation does not form and the redox current thus does not increase. Thus, changes in a measurement of electrical redox current can be used as a signal to interpret changes in concentration of the analyte 180.

The monolayer blocking layer 122 may also be an oxygen limiting layer if properly designed and therefore the sensor may be both an aptamer sensor and an oxygen sensor using the same working electrode 120. However monolayer blocking layers in some cases may only be stable for short periods of time (for example, with mercaptohexanol blocking layers), and hence the permeability to oxygen and therefore diffusion of oxygen may change over time, causing challenges in maintaining an accurate ability to sense oxygen.

As noted more fully herein, certain sensors may require two or more electrodes, reference electrodes, or additional supporting technology or features. Moreover, the sensor can be in duplicate, triplicate, or more, to provide improved data and readings. The measurement of redox current may provide continuous or discrete data and/or readings. Yet further, additional sub-components may be needed for use of the sensor in various applications, (e.g., a reference or counter electrode, a battery, antenna, adhesive, etc.).

### Electrochemical Aptamer Sensor Example 2

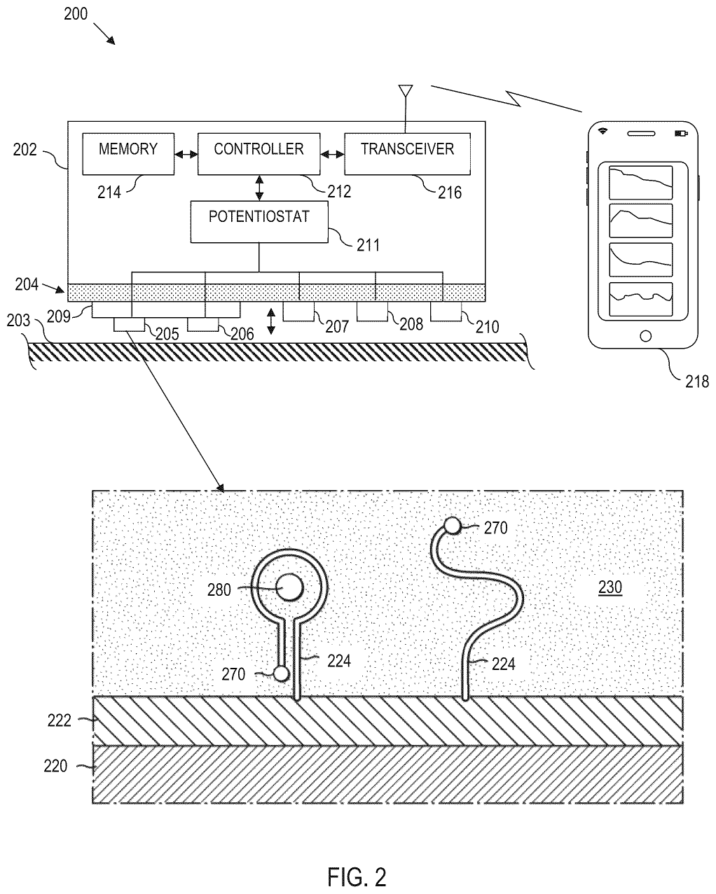

With reference to FIG. 2, a wearable monitoring device is schematically illustrated. In this illustrative example, the wearable monitoring device is implemented as a wearable heart monitoring device 200. However, as will be described more fully herein, the wearable monitoring device can be used for other functions.

The wearable heart monitoring device 200 includes features and processes that are analogous to those of FIG. 1. In this regard, like features or structures are illustrated with a reference number 100 higher than that of FIG. 1. Accordingly, the descriptions provided with regard to FIG. 1 are incorporated into the description of FIG. 2, except where differences are expressly noted.

In this regard, the system of FIG. 2 includes a housing 202 that is intended to attach to the skin 203 of a patient. For instance, the housing 202 can optionally include an adhesive 204 thereon, where the adhesive 204 is provided for securing the housing 202 to a patient wearing the wearable heart monitoring device 200.

The wearable heart monitoring device 200 includes in general, a first electrode 205 configured to measure a first cardiovascular parameter of a patient wearing the heart monitoring device, and a second electrode 206 configured to measure a second cardiovascular parameter of the patient wearing the heart monitoring device, analogous to like elements of FIG. 1.

The wearable heart monitoring device 200 can also include an optional third electrode 207, an optional gel electrode 208, an optional carrier 209, and optional additional sensor(s) 210, etc., analogous to like components of FIG. 1.

Analogous to that of FIG. 1, the housing 202, as schematically illustrated, is coupled to the first electrode 205 and the second electrode 206. Moreover, in some implementations, the housing 202 contains at least a potentiostat 211, e.g., coupled to at least the first electrode 205. The housing 202 can also contain a controller 212 communicably coupled to the potentiostat 211, memory 214 coupled to the controller 212, and a communication interface 216 communicably coupled to the controller 212. Further, in some implementations, the communication interface 216, communicates with a remote processing device 218, such as a smartphone running an app to display results collected and processed by the wearable heart monitoring device 200, as will be described in greater detail herein.

### Operation of an Example Electrode

By way of illustrative example, an exploded view schematically illustrates aspects of a sensor, such as the sensor associated with one or more of the above-described electrodes, e.g., the first electrode 205. Analogous to the wearable heart monitoring device of FIG. 1, the sensor is illustrated as placed in a sample fluid 230, such as subcutaneous interstitial fluid. The sensor includes at least one working electrode 220 (such as gold, carbon, or other suitable electrode material). Additionally, the sensor has at least one blocking layer 222 that may include a plurality of molecules (such as mercaptooctanol or other suitable molecules that are thiol bonded to the electrode). Moreover, as illustrated, at least one aptamer 224 is provided, which is responsive to binding to an analyte 280. Yet further, a redox tag 270 (such as methylene blue), associated with the at least one aptamer 224, such as by being bound thereto. In this placement, a select blocking layer 222 is positioned between the sample fluid 230 and a corresponding working electrode 220. As illustrated, the working electrode 220 lies adjacent to the blocking layer 222.

Analogous to that of FIG. 1, each aptamer 224 includes a redox tag 270 at a distal end thereof, where the distal end is situated in the sample fluid 230.

Different from FIG. 1, in the illustrated implementation, each aptamer 224 couples to a corresponding blocking layer 122, and extends into the sample fluid 230. However, the aptamer 224 need not extend through the corresponding blocking layer 222, and thus, the aptamer 224 does not need to directly contact a corresponding working electrode 220.

In an example implementation, the wearable heart monitoring device 200 includes a non-monolayer blocking layer 222 to which an aptamer 224 is attached. The non-monolayer blocking layer 222 can also be an oxygen limiting layer. The non-monolayer blocking layer 222 is positioned adjacent to at least one electrode 220.

In an example implementation, the non-monolayer blocking layer 222 is formed from one or more materials including, but not limited to, a metal oxide, a semiconductor oxide, a thin polymer film, acrylic, polyamide, an inorganic dielectric, a hydrogel, a fluoropolymer, parylene C, parylene HT, PVDF, silicon dioxide, silicon nitride, titanium dioxide, and barium titanate.

In another example implementation, the non-monolayer blocking layer 222 is selected from acrylic, polyamide, an inorganic dielectric such as SiON, or other suitable blocking material. The example non-monolayer blocking layer 222 shown in FIG. 2 may itself be resistant to fouling, and thus not require a separate antifouling layer, and/or the non-monolayer blocking layer 222 may be allowed to foul (not shown), thereby forming an endogenous anti-fouling layer.

As noted more fully herein, a certain sensor may require two or more electrodes, reference electrodes, or additional supporting technology or features. Moreover, the sensor can be in duplicate, triplicate, or more, to provide improved data and readings. The measurement of redox current may provide continuous or discrete data and/or readings. Yet further, additional sub-components may be needed for use of the sensor in various applications, (e.g., a reference or counter electrode, a battery, antenna, adhesive, etc.).

### Electrochemical Aptamer Sensor Example 3

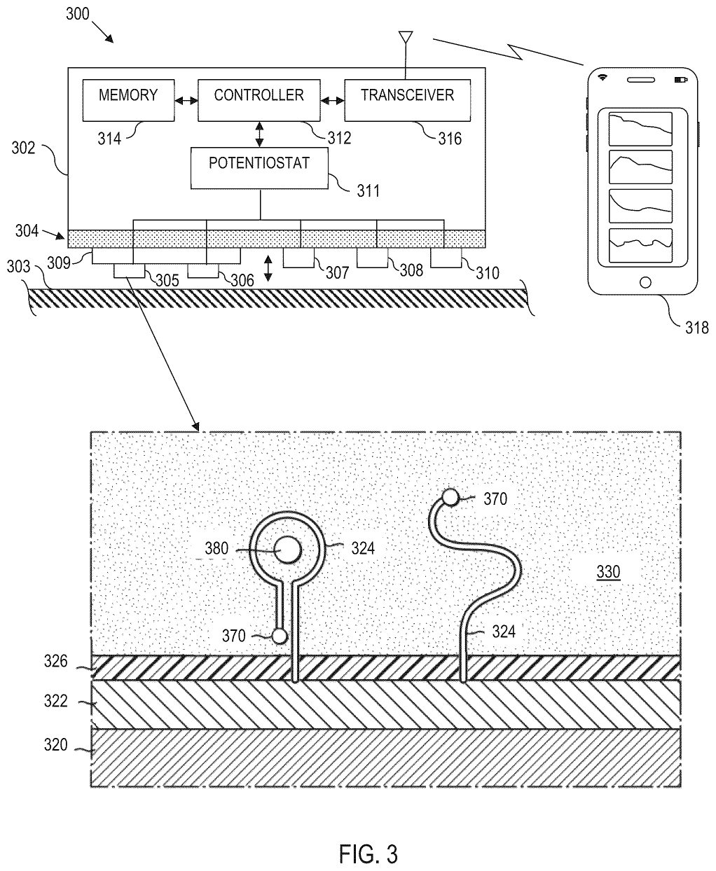

With reference to FIG. 3, a wearable monitoring device is schematically illustrated. In this illustrative example, the wearable monitoring device is implemented as a wearable heart monitoring device 300. However, as will be described more fully herein, the wearable monitoring device can be used for other functions.

The wearable heart monitoring device 300 includes features and processes that are analogous to those of FIG. 1. In this regard, like features or structures are illustrated with a reference number 200 higher than that of FIG. 1. Accordingly, the descriptions provided with regard to FIG. 1 are incorporated into the description of FIG. 3, except where differences are expressly noted.

In this regard, the system of FIG. 3 includes a housing 302 that is intended to attach to the skin 303 of a patient. For instance, the housing 302 can optionally include an adhesive 204 thereon, where the adhesive 304 is provided for securing the housing 302 to a patient wearing the wearable heart monitoring device 300.

The wearable heart monitoring device 300 includes in general, a first electrode 305 configured to measure a first cardiovascular parameter of a patient wearing the heart monitoring device, and a second electrode 306 configured to measure a second cardiovascular parameter of the patient wearing the heart monitoring device, analogous to like elements of FIG. 1.

The wearable heart monitoring device 300 can also include an optional third electrode 307, an optional gel electrode 308, an optional carrier 309, optional additional sensor(s) 310, etc., analogous to like components of FIG. 1.

Analogous to that of FIG. 1, the housing 302, as schematically illustrated, is coupled to the first electrode 305 and the second electrode 306. Moreover, in some implementations, the housing 302 contains at least a potentiostat 311, e.g., coupled to at least the first electrode 305. The housing 302 can also contain a controller 312 communicably coupled to the potentiostat 311, memory 314 coupled to the controller 312, and a communication interface 316 communicably coupled to the controller 312.

In some implementations, the communication interface 316, communicates with a remote processing device 318, such as a smartphone running an app to display results collected and processed by the wearable heart monitoring device 300, as will be described in greater detail herein.

### Operation of an Example Electrode

By way of illustrative example, an exploded view schematically illustrates aspects of a sensor, such as the sensor associated with one or more of the above-described electrodes, e.g., the first electrode 305. Analogous to the wearable heart monitoring device of FIG. 1, the sensor is illustrated as placed in a sample fluid 330, such as subcutaneous interstitial fluid. The sensor includes at least one working electrode 320 (such as gold, carbon, or other suitable electrode material). The sensor also includes at least one aptamer 324 that is responsive to binding to an analyte 380, and a redox tag 370 (such as methylene blue), associated with the at least one aptamer 324, such as by being bound thereto.

The wearable heart monitoring device 300 also includes a non-monolayer blocking layer 322 to which the aptamer 324 is attached. The non-monolayer blocking layer 322 is positioned adjacent to at least one working electrode 320. In an example implementation, the non-monolayer blocking layer 322 is formed from one or more materials, such as those described above with respect to FIG. 2.

The wearable heart monitoring device 300 also includes an antifouling layer 326 that can be positioned adjacent to the non-monolayer blocking layer 322. In an example implementation, the antifouling layer 326 is a solid layer of material or a monolayer terminated with polyethylene glycol attached to non-monolayer blocking layer 322. In another example implementation, the antifouling layer 326 is formed from a zwitterionic material that is bound to layer 322. Membranes such as polybetaine hydrogels may also be added on top of a wearable heart monitoring device 100, 200, 300 (not shown) to prevent fouling.

Thus, as illustrated, the antifouling layer 326 is situated between the sample fluid 330 and the non-monolayer blocking layer 322. Likewise, the non-monolayer blocking layer 322 is situated between the antifouling layer 326 and the at least one working electrode 320.

Analogous to that of FIG. 1 and FIG. 2, each aptamer 324 includes a redox tag 370 at a distal end thereof, where the distal end is situated in the sample fluid 330.

Different from FIG. 1 but analogous to FIG. 2, in the illustrated implementation, each aptamer 324 couples to a corresponding blocking layer 322, and extends into the sample fluid 330. In FIG. 3, the aptamers 324 also pass through the antifouling layer 326. However, the aptamer 324 need not extend through the corresponding blocking layer 322, and thus, the aptamer 324 does not need to directly contact a corresponding working electrode 320.

As noted more fully herein, certain sensors may require two or more electrodes, reference electrodes, or additional supporting technology or features. Moreover, the wearable heart monitoring device 300 can be in duplicate, triplicate, or more, to provide improved data and readings. The measurement of redox current may provide continuous or discrete data and/or readings. Yet further, additional sub-components may be needed for use of the wearable heart monitoring device 300 in various applications, (e.g., a reference or counter electrode, a battery, antenna, adhesive, etc.).

### Multimodal Electrode for Both Analyte Sensing and Oxygen Sensing

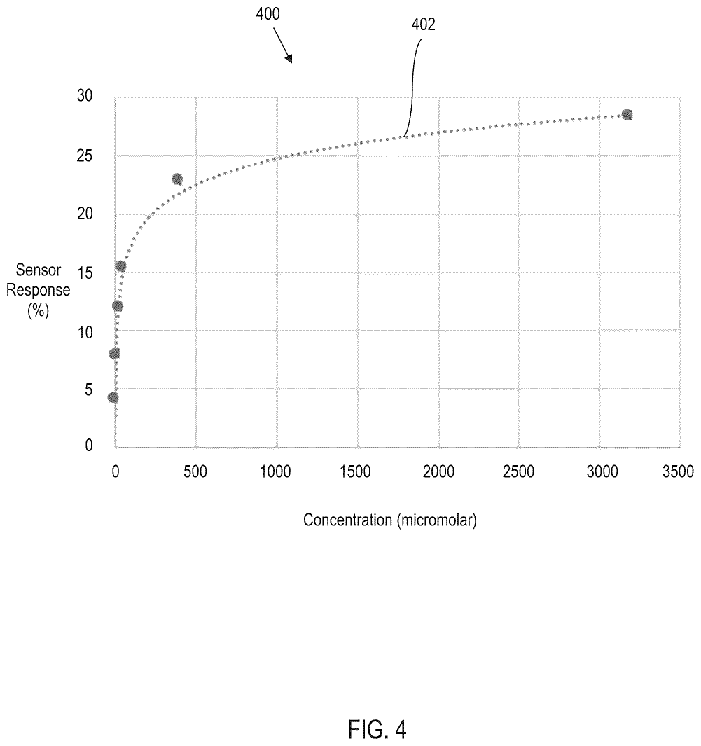

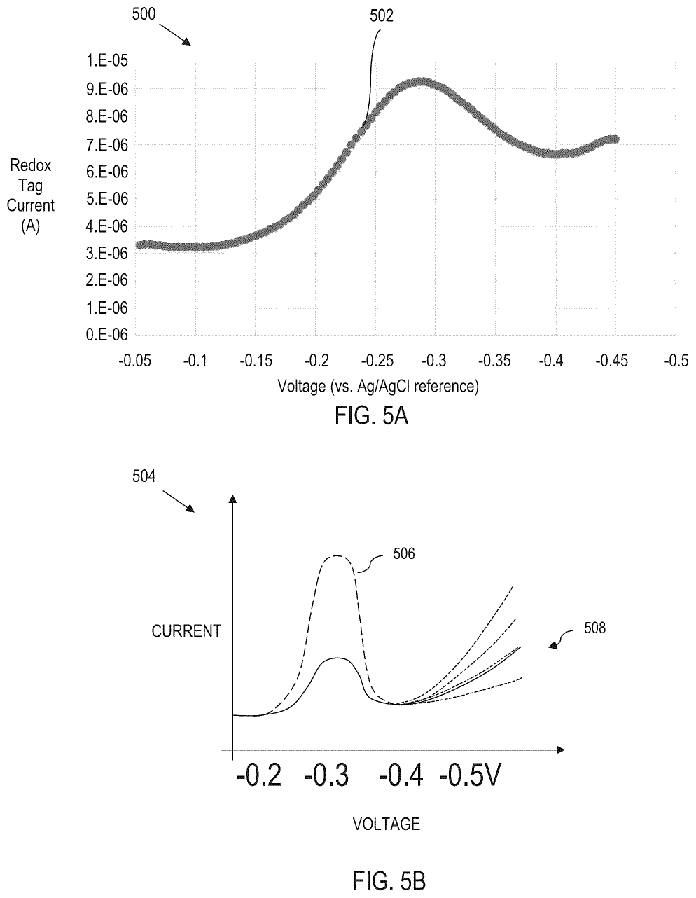

With reference to FIG. 4, FIG. 5A and FIG. 5B, an example demonstration is provided. A gold electrode was further coated with a non-monolayer blocking layer of 2 nm of SiO₂ by e-beam evaporation. A non-monolayer blocking layer was further functionalized with an aptamer for phenylalanine on gold electrode but adapted for placement on SiO₂ with silane chemistry.

Referring initially to an example plot 400 of FIG. 4, the device functions as a sensor as shown in the titration curve 402, using square wave voltammetry measurement.

Referring to the plot 500 as illustrated in FIG. 5A, a methylene blue redox peak is collected (e.g., from the potentiostat and/or controller described above with reference to FIG. 1-FIG. 3) as a square wave voltammogram 502. The square wave voltammogram peak height determines the sensor response (peak height increases over baseline as more phenylalanine is added). At larger negative voltages, such as −0.4 V, the increased background current is due to oxygen reduction of oxygen that diffuses through an oxygen limiting layer, such as may be implemented by the blocking layer 122, 222, 322 (FIG. 1, FIG. 2, FIG. 3, respectively). The magnitude of this oxygen reduction current is dependent on dissolved oxygen concentration in the sensor, and is in a diffusion-limited regime due to the oxygen limiting layer, and is therefore also a dissolved oxygen sensor if the current is sampled at −0.4V or −0.5V or even more negative voltages.

Referring to FIG. 5B, a plot 504 further illustrates this dual measurement where the aptamer and analyte measurement is represented by course dotted line 506, and an oxygen measurement is represented by fine dotted lines 508, which increase in current as oxygen concentration increases. Other measurement techniques such as amperometry, cyclic voltammetry, or other suitable measures can be used to measure sensor response to the analyte and/or to oxygen. As a result, an example implementation includes a device which uses a single electrode to measure at least one target analyte and at least oxygen.

In alternative configurations, an oxygen limiting layer could be placed on a counter electrode. Here, the counter electrode is biased to measure oxygen reduction. Ideally, antifouling chemistry is also placed on the oxygen limiting material when on a counter electrode such that measurement accuracy is preserved in-vivo.

In another alternate example implementation the sensor is an enzymatic sensor such as a second or third generation glucose sensor, and the sensor additionally includes an oxygen limiting layer on the electrode used in such an implementation to enable a device which uses a single electrode to measure at least one target analyte and at least oxygen.

Multimodal Device with Multiple Sensing Modalities

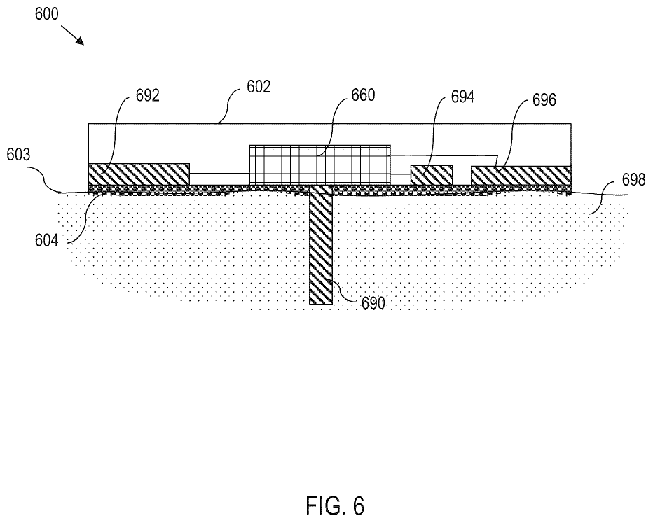

With reference to FIG. 6, a wearable multimodal sensor 600 is illustrated, where like numerals refer to like features three hundred higher than FIG. 3, four hundred higher than FIG. 2, and five hundred higher than FIG. 1. Accordingly, like features are analogous and the discussion of the preceding FIGURES is incorporated into the description of FIG. 6, except where differences are expressly noted.

In an example implementation of the wearable multimodal sensor 600, a housing 602, e.g., a plastic structure, is placed on the skin 603 of a patient. The housing 602 can be placed via a strap, adhesive, etc. For instance, as illustrated, the housing 602 is secured to the skin 603 via adhesive 604. The housing 602 packages a plurality of sensors, including sensor 690, 692, 694, 696 that are operated by electronics 660. As noted more fully herein, the electronics can include a potentiostat, controller, memory, a transceiver, etc.

Sensor 690 may include an analyte-detecting material, an oxygen limiting material (for measuring both the analyte and dissolved oxygen), or both, as noted more fully herein. In this regard, sensor 690 can include an electrode such as electrode 105 and/or electrode 106 (FIG. 1), electrode 205 and/or electrode 206 (FIG. 2), electrode 305 and/or electrode 306 (FIG. 3), and is illustrated extending into sample fluid 698.

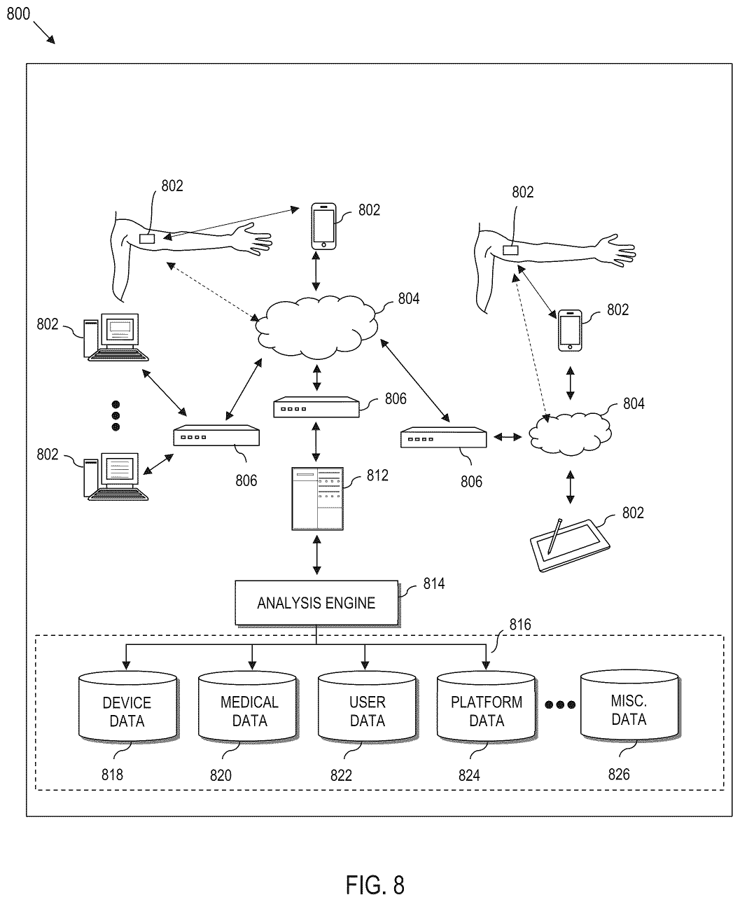

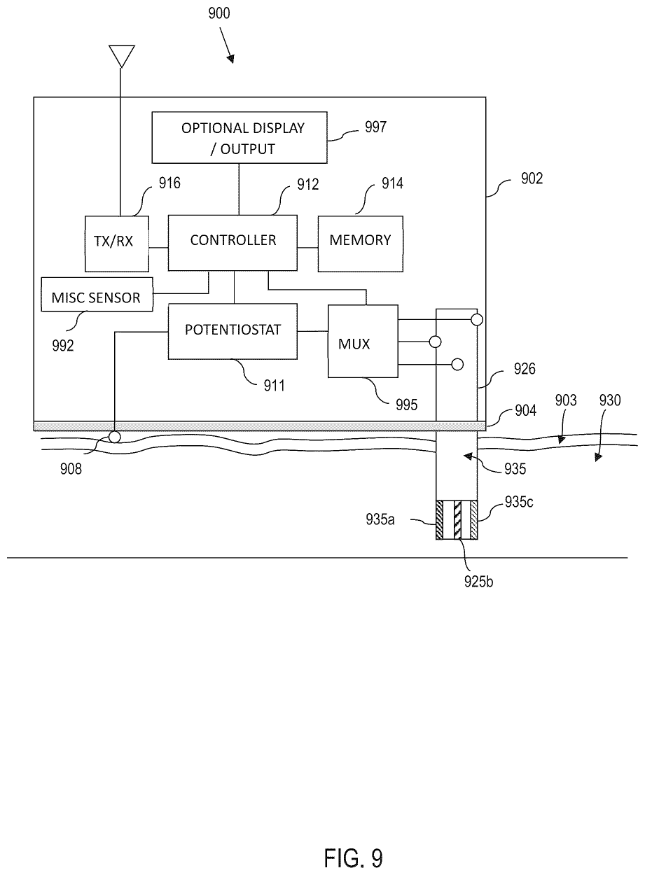

Sensors 692, 694, 696 are each optional, and can include any combination of an optical sensor, a strain sensor, an electrical sensor, and an acceleration sensor for the remaining sensors. In this regard, practical applications can have more than three additional sensors. Moreover, additional description of the wearable multimodal sensor 600 will be detailed with reference to FIG. 8 and FIG. 9.

As noted more fully herein, certain implementations of the wearable multimodal sensor 600 may require two or more electrodes, reference electrodes, or additional supporting technology or features. Moreover, the wearable multimodal sensor 600 can be in duplicate, triplicate, or more, to provide improved data and readings. The measurement of redox current may provide continuous or discrete data and/or readings. Yet further, additional sub-components may be needed for use of the wearable multimodal sensor 600 in various applications, (e.g., a reference or counter electrode, a battery, antenna, adhesive, etc.).

Multicomponent Multimodal Device with Multiple Sensing Modalities

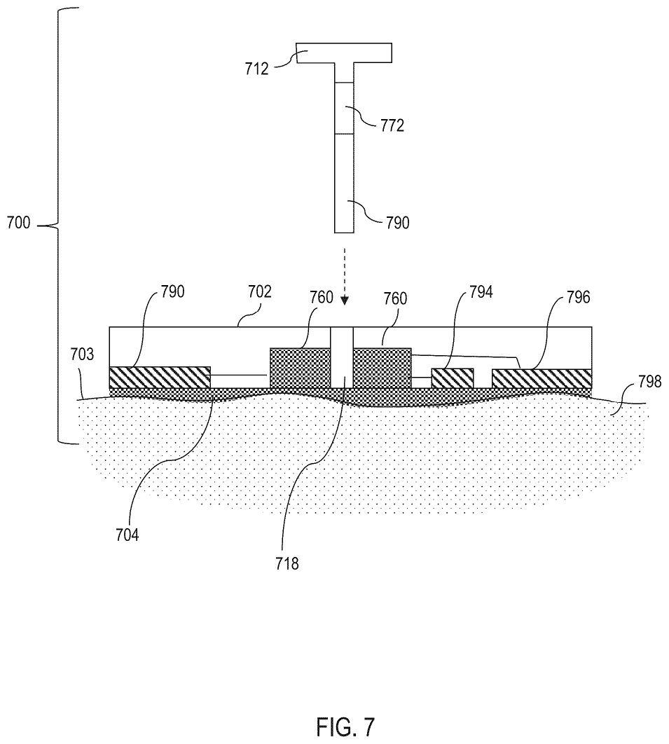

With reference to FIG. 7, a wearable multicomponent multimodal sensor 700 is illustrated, where like numerals refer to like features one hundred higher than FIG. 6, four hundred higher than FIG. 3, five hundred higher than FIG. 2, and six hundred higher than FIG. 1. Accordingly, like features are analogous and the discussion of the preceding FIGURES is incorporated into the description of FIG. 6, except where differences are expressly noted.

In an example implementation, a wearable multicomponent multimodal sensor 700 includes a housing 702, e.g., a plastic structure, that is placed on the skin 703 of a patient. The housing 702 can be placed via a strap, adhesive, etc. For instance, as illustrated, the housing 702 is secured to the skin 703 via adhesive 704. The housing 702 packages a plurality of sensors, including sensor 790, 792, 794, 796 that are operated by electronics 760. As noted more fully herein, the electronics can include a potentiostat, controller, memory, a transceiver, etc.

Sensor 790 may include an analyte-detecting material, an oxygen limiting material (for measuring both the analyte and dissolved oxygen), or both, as noted more fully herein. In this regard, sensor 790 can include an electrode such as electrode 105 and/or electrode 106 (FIG. 1), electrode 205 and/or electrode 206 (FIG. 2), electrode 305 and/or electrode 306 (FIG. 3), and is illustrated extending into sample fluid 798.

Sensors 792, 794, 796 are each optional, and can include any combination of an optical sensor, a strain sensor, an electrical sensor, and an acceleration sensor for the remaining sensors. In this regard, practical applications can have more than three additional sensors. Moreover, additional description of the wearable multimodal sensor 700 will be detailed with reference to FIG. 8 and FIG. 9.

Sensor 790 is illustrated as including an electrical connector 772 for coupling the sensor 790 to electronics 760. Sensor 790 also comprises a housing 712 that is dimensioned to align with an aperture 718 that passes through the housing 702.

In an example implementation, a first sensor can alert that a second sensor should be inserted. For instance, FIG. 7 represents an implementation where the sensor 790 is placed into skin only as needed. By way of example, sensor 790 can implement a first electrode that is initially not used. A second electrode (e.g., defined by one or more of sensors 792, 794, 796) is configured to collect a desired measurement. For instance, sensor 794 can implement an electrocardiogram. Here, the electronics 760 can implement a controller that monitors the output of the second electrode and issues an alert to have the first electrode inserted via the sensor 790 if the monitored output meets a predetermined criterion. For instance, in an example implementation, the sensor 790 can be inserted through the aperture 718 such that the sensor 790 extends into the sample fluid 798. This example can use any insertion mode, including those used in microneedles or continuous glucose meters. However, in practice, insertion of the second sensor can be performed using any suitable technique, even using a separate device co-located on skin.

An example use case scenario is a wearable multicomponent multimodal sensor 700 that monitors ECG for heart failure patients and if a suspected heart degradation or heart attack occurs as measured by the ECG sensor, then another sensor, such as an invasive sensor, (e.g., sensor 790) can be added to measure an analyte such as NT-proBNP or Troponin. As a result, a wearer uses a non-invasive device most of the time and only adds invasive sensor 790 when critically needed. Sensors such as sensor 790 can be added using any suitable method, including the types of sensor inserters used commercially in continuous glucose monitors which rely on a slotted guide (not shown) to place the sensor 790 into the skin 703.

As noted more fully herein, certain implementations of a wearable multicomponent multimodal sensor 700 may require two or more electrodes, reference electrodes, or additional supporting technology or features. Moreover, the wearable multicomponent multimodal sensor 700 can be in duplicate, triplicate, or more, to provide improved data and readings. The measurement of redox current may provide continuous or discrete data and/or readings. Yet further, additional sub-components may be needed for use of the wearable multicomponent multimodal sensor 700 in various applications, (e.g., a reference or counter electrode, a battery, antenna, adhesive, etc.).

### System Overview

With a basic understanding of the above-described sensors in place, reference is now drawn to FIG. 8, which illustrates a general diagram of a computer system 800 according to various aspects of the present disclosure.

The computer system 800 comprises a plurality of hardware processing devices (designated generally by the reference 802) that are linked together by one or more network(s) (designated generally by the reference 804).

The network(s) 804 provides communications links between the various processing devices 802 and may be supported by networking components 806 that interconnect the processing devices 802, including for example, routers, hubs, firewalls, network interfaces, wired or wireless communications links and corresponding interconnections, cellular stations and corresponding cellular conversion technologies (e.g., to convert between cellular and TCP/IP, etc.). Moreover, the network(s) 804 may comprise connections using one or more intranets, extranets, local area networks (LAN), wide area networks (WAN), wireless networks (Wi-Fi), the Internet, including the world wide web, cellular and/or other arrangements for enabling communication between the processing devices 802, in either real time or otherwise (e.g., via time shifting, batch processing, etc.).

A processing device 802 can be any device capable of communicating with another processing device 802, e.g., via Bluetooth, ultrawide band, near field communication (NFC), via one or more radio frequencies (RF) or via any other form of wired or wireless communication, over the network 804, or combinations thereof.

Some examples of processing devices 802 are cellular devices (including cellular mobile telephones (i.e., smartphones)), tablet computers, netbook computers, notebook computers, personal computers, servers, cloud devices, edge devices, etc.

Also, in certain contexts and roles, a processing device 802 is intended to be a wearable monitoring device. Examples of a wearable monitoring device include a purpose-driven appliance,

Internet of Things (IoT) device, special purpose device, etc. A processing device 802 implemented as a wearable monitoring device is schematically illustrated in FIG. 8 as a wearable device mounted to a patient's arm solely for convenience of illustration. In practical applications, the wearable monitoring device can attach to other parts of a patient's body. Regardless, the wearable monitoring device shown as a processing device 802 within the computer system 800 can include features, structures, and processes analogous to any combination of that described in the preceding FIGURES.

In some implementations, the wearable monitoring device can communicate locally (e.g., to a smart phone) via Bluetooth, ultrawide band, via one or more radio frequencies (RF) or via any other form of wired or wireless communication. In other implementations, the wearable monitoring device can communicate across a network, e.g., via Wi-Fi and/or communicate locally to another processing device 802.

The illustrative computer system 800 also includes a processing device implemented as a server 812 (e.g., a web server, file server, and/or other processing device) that supports an analysis engine 814 and corresponding data sources (collectively identified as data sources 816). The analysis engine 814 and data sources 816 provide the resources to implement and store data related to collecting and aggregating data from wearable monitoring devices, captured events, combinations thereof, etc., as described in greater detail herein.

In an exemplary implementation, the data sources 816 are implemented by a collection of databases that store various types of information. Solely by way of example, the data sources 816 can include device data 818, e.g., data related to wearable monitoring devices, including configuration data, version data, software versioning and control, data generated from wearing a wearable monitoring device, etc. The data sources 816 can also include medical data 820, e.g., medical research, etc., used to calibrate, tune, design, modify, etc., wearable monitoring devices. The data sources 816 can also optionally include user data 822, e.g., data regarding the patients that are wearing the wearable monitoring devices, where such data is collected. As yet further examples, the data sources 816 can include platform data 824, e.g., data used by the analysis engine 814, e.g., computer drivers, GUI information, algorithms for processing physiological conditions, etc. As yet a further example, the data sources 816 can include miscellaneous data 826, e.g., any data needed by the analysis engine 814 that is not otherwise accounted for above.

Considering FIG. 8 as an environment used by wearable monitoring devices, in some implementations, the processing of physiological data of a corresponding patient wearing the wearable monitoring device (e.g., biochemical sensing with additional sensing modalities that enhance patient care or health and wellness) can be carried out entirely on a processing device 802 (such as a wearable monitoring device itself); on a processing device 802 such as a smartphone, by the analysis engine 814, or via combinations thereof (e.g., by distributing processing tasks among two or more processing devices).

With specific regard to a processing device 802 implemented as a wearable monitoring device (see processing device 802 schematically attached to a patient's arm), it may be desirable to carry out all of the processing on the wearable monitoring device itself. In this regard, a corresponding device such as a smartphone can optionally provide a graphical user interface for displaying dashboard measurement results, but all processing is carried out on the wearable monitoring device itself.

In other implementations, the smart phone can carry out some processing, e.g., to compare computed data to dashboard thresholds, to carry out algorithms, rules, or other processing, as described more fully herein.

In still other implementations, the analysis engine 814 can collect data from each wearable monitoring device, e.g., for trend analysis of patient data, for device state of health monitoring (e.g., to detect faults in the wearable devices themselves), for battery charge level monitoring, for versioning (such as to carry out software updates), etc.

In some implementations, the analysis engine 814 is controlled by a third party, e.g., the manufacturer of the wearable monitoring devices that are implemented in the environment.

In some implementations, the analysis engine 814 schematically represents integration into an electronic health record system, e.g., to connect a patient to the patient's doctor so that the doctor can access the electronic data generated by a corresponding wearable monitoring device.

### Example Monitoring Device

Referring now to FIG. 9, an example wearable monitoring device 900 is schematically illustrated, according to aspects of the present disclosure. The wearable monitoring device 900 can represent an example implementation of a processing device 802 (FIG. 8), e.g., a wearable monitoring device implemented using any one of the wearable multimodal sensors described with regard to the previous FIGURES. In this regard, the wearable monitoring device 900 can include features, structures, and processes analogous to any combination of that described in the preceding FIGURES. In particular, the sensor/electrode technology described with reference to FIG. 1-FIG. 7 can be integrated into the wearable monitoring device 900 in any combination. In this regard, like features or structures are illustrated with a reference number 800 higher than that of FIG. 1, 700 higher that that of FIG. 2, 600 higher than that of FIG. 3, 300 higher than that of FIG. 6, 200 higher than that of FIGS. 7, and 100 higher than that of FIG. 8. Accordingly, the descriptions provided with regard to FIG. 1-FIG. 8 are incorporated into the description of FIG. 9, except where differences are expressly noted.

The wearable monitoring device 900 includes a housing 902 that attaches to a patient. For instance, the housing 902 can attach to the skin 903 (epidermis) of the patient via an adhesive 904, a strap, or other securement.

The wearable monitoring device 900 also includes at least one electrode 935. In this regard, FIG. 9 incorporates the disclosures of FIG. 1-FIG. 7 in any combination into the wearable monitoring device 900 of FIG. 9. For instance, one or more electrodes may penetrate the skin 903 and enter a sample fluid 930, such as subcutaneous interstitial fluid of the patient. As noted more fully herein, the electrode may penetrate the skin at the time of application, or when triggered by a measured condition, as described more fully herein.

For instance, FIG. 9 illustrates at least a first electrode 935a and may include a second electrode 935b and further may include a third electrode 935c. In some implementations, there can be more than three electrodes. In some implementations, one or more electrodes include an analyte detecting material, e.g., aptamers or enzymes, such that continuous sensing can be carried out. For instance, the first electrode 935a, second electrode 935b, third electrode 9935c, or combinations thereof can be implemented by electrode 105 and/or electrode 106 (FIG. 1); electrode 205 and/or electrode 206 (FIG. 2); electrode 305 and/or electrode 306 (FIG. 3); sensor 690 (FIG. 6); sensor 790 (FIG. 7), or combinations thereof.

In practical applications, the housing 902 is couplable to the electrode(s) 935. As used herein, “couplable” is to be construed broadly to mean any one of permanently coupled, detachably coupled, temporarily coupled, user attachable, user detachable, user attachable and detachable, factory attachable, factory detachable, factory attachable and detachable, or any combination thereof, unless specifically noted otherwise.

In an example implementation, the first electrode 935a defines a sensor (e.g., an aptamer sensor) that is configured to measure at least one of b-type natriuretic peptide (BNP), or N-terminal pro b-type natriuretic peptide (NT-proBNP). In some implementations, the first electrode 935a may also optionally be configured to measure Troponin. Thus, in this example, the first electrode 935a can be implemented in a manner analogous to the first electrode 105 (FIG. 1); first electrode 205 (FIG. 2); first electrode 305 (FIG. 3), etc.

The second electrode 935b, where provided, is configured to measure at least one of parameter associated with heart disease or heart disease treatment of a patient wearing the heart monitoring device. For instance, in some implementations, the second electrode 935b is configured to measure at least one of potassium, creatinine, or Troponin. Thus, in this example, the second electrode 935b can be implemented in a manner analogous to the second electrode 106 (FIG. 1); second electrode 206 (FIG. 2); second electrode 306 (FIG. 3), etc.

In some embodiments, the first electrode 935a, the second electrode 935b, or both can be configured to measure tissue oxygen desaturation, oxygen concentration, etc.

In some implementations, the first electrode 935a is configured to measure NT-proBNP, the second electrode 935b is configured to measure potassium, and the third electrode 935c is configured to measure creatinine.

The wearable monitoring device 900 may include at least one additional sensor 992 such as the other sensing modalities taught herein. For instance, sensor 992 can comprise an optical sensor (e.g., an optical heart rate sensor, an optical oxygen saturation sensor, etc.), an accelerometer, an acceleration measuring or inferring sensor, an electrical sensor, an electrocardiogramansor, an optical pulse wave sensor configured for measuring blood pressure, an RF-measured thoracic-fluid index sensor, a strain sensor, an electrical sensor, a mechanical sensor, combinations thereof, etc.

As such, the location of the additional sensor 992 may, in practice, be different from the schematically illustrated location, based for example, on sensor function and measurement type. In this regard, the illustrated location of the additional sensor 992 is purely for convenience and simplicity of illustration.

As noted more fully herein, e.g., with regards to the preceding FIGURES and the corresponding discussion herein, which is incorporated by reference into FIG. 9, the first electrode 935a and/or the second electrode 935b of the wearable monitoring device 900 can be formed from a carrier that defines a generally flat structure having a first surface and an optional second surface opposite the first surface. For example, the first electrode 935a can be formed on the first surface and the second electrode 935b can be formed on the second surface. As another example, using photolithography, screen printing pattering, or other suitable methods the first electrode 935a and the second electrode 935b can be patterned on the first surface, second surface, or combination thereof.

The third electrode 935c or additional electrodes (where utilized) can likewise be integrated as taught using one or more of the above examples.

As illustrated, the housing 902 includes a potentiostat 911 that is communicably coupled to at least one electrode, e.g., the electrode 935a, 935b, 935c (or a combination thereof) using an optional multiplexer 995, or alternatively each of electrodes 935a, 935b, 935c can receive a direct dedicated connection to the potentiostat 911. In practical applications, the term “potentiostat” is to be interpreted broadly, and is not limited to any particular number of sensors. For instance, the potentiostat can be implemented as a bipotentiostat, polypotentiostat, etc., depending upon the sensor configuration provided by the wearable monitoring device 900.

Additionally, the wearable monitoring device 900 includes a controller 912 that is communicably coupled to memory 914. The controller 9112 is also communicably coupled to a communication interface 916.

The controller 912 includes necessary electronics that enable the controller 912 to carry out the intended functionality of the wearable monitoring device. For instance, the controller 912 can include a processor, bus interface, ports, registers, memory, etc., that enables the wearable monitoring device 900 to carry out the functionality described more fully herein.

Also, as illustrated, the controller 912 is communicably coupled to one or more of the optional multiplexer 995, potentiostat 911, the memory 914, the transceiver(s) 916, optional miscellaneous sensors 992, optional display/output 997, combinations thereof, etc.

The communication interface 916 may comprise, for example, at least one transceiver that communicates via Bluetooth, Wi-Fi, ultrawide band, near field communication, combinations thereof, etc.

The optional display/output 997 can comprise a display screen, a dimensionally limited display screen, LED, a touch screen, a haptic output, a light output, a speaker/alarm, or combinations thereof.

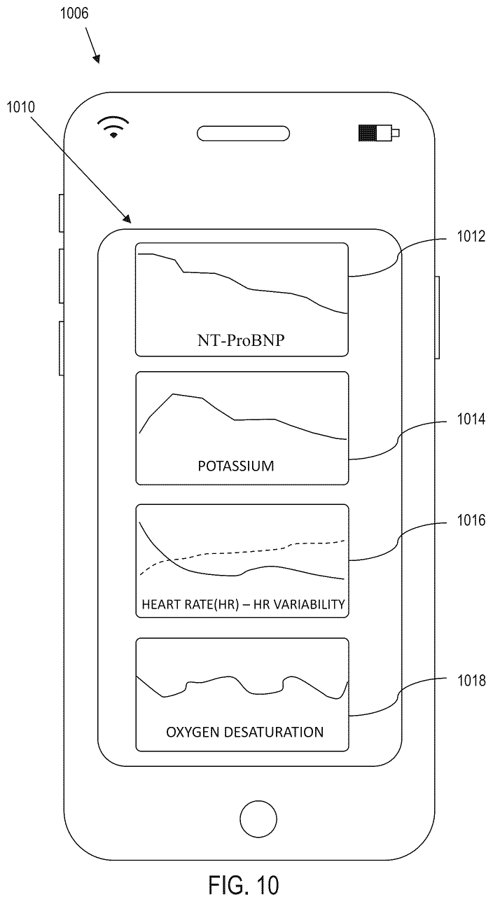

The controller 912 uses the potentiostat 911 to collect measurements from electrode(s), e.g., electrodes 935a, 935b, 935c, and stores the collected measurements in the memory 914. The controller 912 may further provide filtering, analysis, control, authorization, authentication, and other controller specific functions. The communication interface 916 facilitates coupling the wearable monitoring device 900 with an external computing device, e.g., a smartphone, a cloud computer, etc. In this regard, the communication interface 916 can include one or more modalities, each with different data and/or authorizations. For instance, a patient may access data from the wearable monitoring device 900 on a processing device 802 (FIG. 8), smartphone 1006 (FIG. 10), whereas a doctor may be able to access more detailed information from a cloud server and/or through electronic health records (see server 812, analysis engine 814; FIG. 8). In this regard, multiple modalities of communication may be utilized with wearable monitoring device 900.

In some implementations, the adhesive 904 of the wearable monitoring device 900 is, or includes, a gel pad electrode 908 that is connected to at least one of the potentiostat 911, the controller 912, or the sensor 992. For example, a gel pad electrode 908 could be the counter or reference electrode for the electrodes 935a, 935b, 935c, a potentiometric sensor for heart rate or ECG, or other sensing modalities as taught herein.

In some implementations, the wearable monitoring device 900 is constructed from at least three sensors (e.g., including electrode 935a, 935b, 935c, multiple instances of sensor 992, combinations thereof, etc.), a select one of the three sensors for simultaneous monitoring by the controller, of breathing rate, blood oxygen saturation (SpO₂), and tissue oxygenation so as to provide a measure of pulmonary-cardio-vascular performance.

Additional details for sensors/electrodes described above with regard to the preceding FIGURES are now provided.

### NT-ProBNP

As noted above, the first electrode 935a can function as an aptamer sensor (e.g., using any combination of the features of FIG. 1-FIG. 3, FIG. 6 or FIG. 7) that measures N-terminal pro b-type natriuretic peptide (NT-proBNP). For instance, a very high-risk period for patients hospitalized for heart failure is when transitioning from the hospital to home, which defines a period exacerbated by challenges in drug titration. If the treatment is up-titrating the drugs and the NT-proBNP measurement is decreasing, then the patient is recovering properly. Analogously, if the NT-proBNP measurement is not decreasing, then this can serve as an indicator of complications with, or non-response to, treatment. An example NT-proBNP aptamer is the following sequence or variants of it:

GGTCGTAGTGGAAACTGTCCACCGTAGACCGGTTATCTA

The example NT-proBNP aptamer is sensitive and selective even at 10's to 100's of pM in whole blood, even when being operated as a continuous sensing and/or monitoring format. Additional such aptamers are available commercially from companies such as Somalogic and BasePair Bio.

### Potassium

In some implementations, the second electrode 935b can function as a potassium aptamer sensor (e.g., using any combination of the features of FIG. 1-FIG. 3, FIG. 6 or FIG. 7). The potassium sensor herein can measure potassium levels, which serve as a strong predictor of all-cause mortality. For instance, greater than 60% of newly diagnosed heart failure patients are out of range in their potassium levels, and about 40% of newly diagnosed heart failure patients are hyperkalemic. For heart failure with preserved ejection fraction (50% of HF), during treatment with mineralocorticoid antagonists, monitoring of potassium can be utilized to reduce the risk of hyperkalemia. In this regard, rapid medication up-titration can increase the occurrence of adverse events such as hyperkalemia. The potassium (K+) sensor may utilize aptamers that are very small in size for a robust response and reduced susceptibility to interferents (minimally-responsive to other electrolytes):

5′-CCAACGGTTGGTGTGGTTGG-3′
5′-CCAAGGTTGGTGTGGTTGG-3′
5′-ACCAAGGTTGGTGTGGTTGG-3′
5′-CCCAAGGTTGGTGTGGTTGG-3′
5′-CAAGGTTGGTGTGGTTGG-3′
5′-GGTTGGTGTGGTTGG-3′
5′-AAAATGAGGGAGGGG-3′
5′-AAAATGGACAAACGA-3′
5′-AAAAGGGTTAGGGTTAGGGTTAGGGAAAAGCGTCCTCCG-3′
5′-AAAACGGAGGACGC-3′
5′-AAAAGGGTTAGGGTTAGGGTTAGGG-3′.

An alternate strategy for K+ is a solid-state ion-selective electrodes for K+. But as noted previously using similar sensing modalities on electrode 935b is beneficial and therefore ion-selective electrodes are less desirable than an aptamer sensor for K+.

Sensor 935b when measuring potassium can measure and report one or more of the following: a continuous measure of potassium plotted vs. time over minutes, hours, days, months, or years; a score of % time in range for example of 3.6 to 5.2 mM potassium and the score provided on an ongoing basis or on an hourly, semi-daily, daily, multiday, weekly, biweekly, monthly, or yearly format; an alert when potassium falls out of range such as outside of 3.6 to 5.2 mM potassium to alert the user to a poor diet choice or to take potassium altering substances such as potassium supplements or rich foods for hypokalimia or to administer calcium gluconate, beta-agonists, baking soda, exchange resin, furosemide, or other treatments for hyperkalemia.

### Creatinine

Renal dysfunction is common in patients with heart failure and is associated with high morbidity and mortality. Cardiac and renal dysfunction may worsen each other through multiple mechanisms such as fluid overload and increased venous pressure, hypo-perfusion, neurohormonal and inflammatory activation, and concomitant treatment. Many drugs used for the treatment of HF may influence renal function. Short-term changes in serum creatinine must be distinguished from long-term changes, which may be associated with nephron loss and permanent renal impairment. Creatinine may use one of several aptamers that are available commercially through Dianox (Denmark) or Creative Biolabs (USA) or disclosed in WO2017210683A1 “Aptamer-based analyte assays”. The normal level of creatinine in the blood of a healthy individual is 40-150 μM, but it can exceed 1,000 μM in certain extreme circumstances. Patients with severe renal deficiency could have creatinine levels greater than 500 μM and may eventually require dialysis or kidney replacement. Renal injury or dysfunction typically occurs with a serum creatinine increase by 10's of percent or 50% from baseline within 7 days, or an absolute increase in creatinine of 10's μM over 48 h. In this regard, the third electrode 935c can function as a creatinine aptamer sensor.

### Oxygen Desaturation

In some implementations herein, an oxygen desaturation measurement added by a dual-reading from the same sensors that measure analytes using electrode or electrodes (e.g., any one or more of electrode 935a, 935b, 935c), e.g., using any combination of the features of FIG. 1-FIG. 3, FIG. 6 or FIG. 7). Oxygen desaturation can be used to detect sleep disordered breathing conditions such as obstructive sleep apnea (OSA), which can influence patient-centered outcomes. For instance, some implementations herein can detect desaturation events and calculate a resulting desaturation index, which can be relevant to over 65% of heart failure patients. In this regard, tissue oxygenation measurements may have greater clinical value than blood oxygenation, especially for heart failure patients with peripheral artery disease.

Solely by way of example, in an implementation, a sensor can be calibrated or batch calibrated in a beaker of serum that is degassed, where oxygen is slowly added. As an alternative, a beaker of serum could start as is with dissolved oxygen and then be slowly degassed. The oxygen read by the sensor can be compared to a gold standard reference devices such as Clark-electrode or assay.

Moreover, another example implementation provides a monitoring device that simultaneous monitors of breathing rate, blood oxygen saturation (SpO₂), and tissue oxygenation can provide a powerful and complete measure of pulmonary-cardio-vascular performance in chronic disease patients. Normal oxygen saturation should be 96% to 97%. A drop below 90% is considered mildly abnormal, while 80% to 89% is considered moderately abnormal. A drop below 80% is considered severely abnormal. An oxygen desaturation index score is based on how much and how often oxygen level drops during the test. The degree of change from baseline can be measured in two different ways with the following criteria:

According to guidelines from the American Academy of Sleep Medicine, any respiratory event during sleep with a 3% drop in blood oxygen levels is counted towards the total. For example, a change from 95% to 92% would be an event that is counted toward the index's total.

According to Medicare (and some other insurances that still rely on older scoring rules), they require a 4% change for an event to be counted toward the index. Aspects herein may therefore include measurement, recording and/or reporting of oxygen desaturation index.

### Heart Rate

According to some implementations herein, a measuring device can include a sensor that measures at least one of optical heart rate, heart rate variability, or ECG.

Heart rate measurements can be carried out by measuring voltages between a first electrode inside the body (935) and an second electrode outside the body (e.g., gel pad electrode 908, sensor 992, etc.). Heart rate data can be compared to known electrical models for heart activity. Because the skin's stratum corneum forms an externally-facing electrical capacitor, the wearable monitoring device 900 can pick up heart rate by measuring the time-lag or electrical phase-shift between R-waves regardless of placement position on the body. According to some aspects, additional electrodes on the skin surface may be added to measure ECG, like the Zio AT ECG patch which has two electrodes, e.g., distanced by 5 centimeters (cm) on the skin, or like other electrode formats used in the field of heart rate or ECG measurements.

### Example Configurations

In some implementations, the first electrode 935a is configured to measure at least one of b-type natriuretic peptide (BNP) or N-terminal pro b-type natriuretic peptide (NT-proBNP) and the second electrode 935b is configured to measure at least one of potassium, creatinine or Troponin. Additionally, the first electrode 935a or the second electrode 935b can be configured to measure oxygen concentration.

As another example, the first electrode 935a and/or the second electrode 935b can function as a standby sensor for detecting a heart attack by measuring Troponin.

As noted above, the sensor 950 can comprise a gel electrode. Here, with respect to the first electrode 935a and/or the second electrode 935b, the gel electrode is utilized for heart rate or ECG monitoring, and functions as a counter and/or reference for the first electrode 935a and/or the second electrode 935b.

The first electrode 935a and the second electrode 935b can also optionally be provided on a carrier, where the carrier projects outwardly from the housing such that when applied to the patient, the gel pad electrode of the housing is operatively configured to be adherable outside the body, whereas the first electrode 935a and the second electrode 935b puncture the skin so as to reside inside the body.

In some implementations, wearable monitoring device 900 can further comprise at least one additional sensor selected from an accelerometer, an electrocardiogramansor, an optical pulse wave sensor configured for measuring blood pressure, or an RF-measured thoracic-fluid index sensor.

In some implementations, the wearable monitoring device 900 can include at least three sensors for simultaneous monitoring by the controller, of breathing rate, blood oxygen saturation (SpO2), and tissue oxygenation so as to provide a measure of pulmonary-cardio-vascular performance.

In some implementations of the wearable monitoring device 900, a second sensor 995 can alert that a first sensor 935 should be inserted. Insertion of the first sensor 935 can be performed using any suitable technique, even a separate device co-located on skin. By way of a non-limiting but illustrative example, the first electrode 935a is invasive and is initially not used. The second electrode 995 is non-invasive, and is configured as an electrocardiogram. Here, the controller monitors the output of the second electrode 995 and issues an alert to have the first electrode 935a installed/inserted if the monitored output meets a predetermined criterion. The first electrode 935a can be inserted through an aperture in the housing, or other techniques can be applied.

In some implementations, the housing further comprises an aperture therethrough. Here, the first electrode is invasive and is initially not used. Correspondingly, the second electrode is non-invasive, and is configured as an electrocardiogram. Here, the controller monitors the output of the second electrode and issues an alert to have the first electrode installed via the aperture in the housing if the monitored output meets a predetermined criterion, e.g., the electrocardiogram data yields results that are indicative of a patient at risk for having a heart condition such as a heart attack.

In some implementations, the controller is operatively configured to collect heart analytical data based upon measurements collected from the first electrode and the second electrode, and store the results in memory, and the controller is further operatively configured to extract the heart analytical data from the memory and convey the data to a remote processing device.

### Example Wearable Heart Monitoring Device

According to yet further aspects herein, a wearable monitoring device 900 is provided. The wearable monitoring device 900 comprises a housing 902 having an adhesive 904 thereon. The adhesive 904 is provided for securing the housing 902 to a patient wearing the heart monitoring device. The wearable monitoring device 900 also comprises a first electrode 935a configured to measure a first cardiovascular parameter of a patient wearing the heart monitoring device, a second electrode 935b (or electrode 955) configured to measure a second cardiovascular parameter of the patient wearing the heart monitoring device, and an optional third electrode (e.g., a select one of the gel pad electrode 908, sensor 992) functioning as a counter electrode for at least one of the first electrode 935a and the second electrode 935b. Under this configuration, the first electrode 935a and the second electrode 935b are operationally configured as at least one needle electrode that is capable of puncturing through an epidermis of the patient, and the third electrode is operationally configured to attach to the outer skin of the patient. Moreover, the housing 902 contains at least a potentiostat 911 that couples to at least the first electrode 935a, a controller 912 communicably coupled to the potentiostat 911, memory 914 coupled to the controller 912, and a communication interface 916 communicably coupled to the controller 912.

In this regard, the first electrode 935a may be configured to measure the first cardiovascular parameter of the patient by measuring at least one of b-type natriuretic peptide (BNP) or N-terminal pro b-type natriuretic peptide (NT-proBNP). Analogously, the second electrode 935b may be configured to measure the second cardiovascular parameter of the patient by measuring at least one of potassium, creatinine or Troponin. Also, in some implementations, the first electrode 935a or the second electrode 935b is configured to measure oxygen concentration. In some implementations, the third electrode further measures at least one of an electrocardiogra measurement, heart rate (HR), heart rate variability (HRV), HR+HRV, Oxygen desaturation, blood pressure, breathing rate, or combinations thereof.

In some implementations, the controller 912 adjusts a voltage scanning window thus eliminating the need for a reference electrode.

According to still further aspects herein, a wearable monitoring device 900 comprises a first electrode 935a and a housing 910. The first electrode 935a measures at least one target analyte and at least one oxygen characteristic. The housing 910 is coupled to the first electrode 935a. Also, the housing 910 contains at least a potentiostat 991 coupled to the first electrode 935a, a controller 993 communicably coupled to the potentiostat 991, memory 992 communicably coupled to the controller 993, and a communication interface 994 communicably coupled to the controller 993. In use, the potentiostat 991 collects a measurement from the first electrode 935a, the controller 993 stores the measurement from the potentiostat 991 in memory 992, and the controller 993 wirelessly communicates the measurement via the communication interface 994.

As described more fully herein, the first electrode 935a can comprise an aptamer sensor further configured for measuring the at least one oxygen characteristic (see also, the discussion herein with regard to FIG. 1-FIG. 7). Also, the first electrode 935a can comprise an enzymatic sensor having an oxygen limiting layer on the electrode such that a single electrode measures the at least one target analyte and the at least one oxygen characteristic.

The wearable monitoring device 900 according to this configuration can also optionally have at least one additional electrode 995 comprising a select one of an optical sensor, a strain sensor, an electrical sensor, and an acceleration sensor.

### Example Wearable Heart Monitoring Device

According to additional aspects herein, a wearable monitoring device 900 comprises a first electrode 935a that measures at least one target analyte, a counter electrode, such as gel pad electrode 908 and/or sensor 992 biased to measure oxygen reduction, and a housing 902 coupled to the first electrode 935a. The housing 902 contains at least a potentiostat 911 communicably coupled to the first electrode 935a, a controller 912 communicably coupled to the potentiostat 911, memory 914 communicably coupled to the controller 912, and a communication interface 916 communicably coupled to the controller 912. In use, the potentiostat 911 collects a measurement from the first electrode 935a, the controller 912 stores the measurement from the potentiostat 911 in memory 914, and the controller 912 wirelessly communicates the measurement via the communication interface 916.

In some implementations, a counter electrode comprises antifouling chemistry on the oxygen limiting material so as to preserve measurement accuracy in-vivo. The monitoring device of this example implementation can also optionally include at least one additional electrode 992 comprising a select one of an optical sensor, a strain sensor, an electrical sensor, and an acceleration sensor.

### Example Wearable Heart Monitoring Device

According to still further aspects herein, a wearable monitoring device 900 comprises a first sensor, such as gel pad electrode 908 and/or sensor 992, implementing non-invasive sensor, and a housing 902 having an aperture therethrough (see for example, 718, FIG. 7). The housing 902 contains at least a potentiostat 911 communicably coupled to the first sensor, a controller 912 communicably coupled to the potentiostat 911, memory 914 communicably coupled to the controller 912, and a communication interface 916 communicably coupled to the controller 912. In use, the potentiostat 911 collects a measurement from the first sensor, the controller 993 stores the measurement from the potentiostat 991 in memory 992, and the controller 912 wirelessly communicates the measurement via the communication interface 916. Moreover, the controller 912 detects at least one condition based upon the measurements from the first sensor, and upon the controller detecting the at least one condition, the controller 912 provides a signal to trigger a second sensor 935a to be added to the monitoring device. The second sensor 935a defines an invasive sensor that is inserted through the aperture of the housing 902, the second sensor 935a measuring an analyte.

By way of example, the first sensor an comprise an electrocardiogramansor, the at least one condition comprises a heart condition of a patient wearing the monitoring device, and the second sensor 935a measures an analyte comprising at least one of NT-proBNP or Troponin.

### Dashboards

Referring to FIG. 10, a smart phone 1006 can run an optional app that provides a graphical user interface 1010 for displaying data collected or otherwise computed by the controller 912 of the wearable monitoring device 900 (FIG. 9). Data can also be derived from the processing device 802 attached to a patient's arm (FIG. 8). Yet further, the graphical user interface 1010 can display data collected by the controller 112 (FIG. 1); controller 212 (FIG. 2); controller 312 (FIG. 3); electronics 660 (FIG. 6); or electronics 760 (FIG. 7). For instance, solely by way of example, the graphical user interface 1010 can show graphs of NT-proBNP 1012, potassium 1014, heart rate and/or heart rate variability 1016, oxygen desaturation 1018, or any combination of measures as described more fully herein.

In some implementations, the smart phone can pull data from the wearable monitoring device (e.g., wearable heart monitoring device 100 (FIG. 1); wearable heart monitoring device 200 (FIG. 2); wearable heart monitoring device 300 (FIG. 3); wearable multimodal sensor 600 (FIG. 6); wearable multimodal sensor 700 (FIG. 7); processing device 802 (FIG. 8); wearable monitoring device 900 (FIG. 9) for display on the graphical user interface 1010. In other implementations, the wearable monitoring device can push data to the smart phone.

In some implementations, the smart phone merely serves as a visual interface to display data as a graph, dashboard, visual metaphor, alert, etc.

### Example Use Case Scenario

In view of the above, aspects herein provide a multi-modal (multi-channel) wearable cardiac monitoring device that is specifically well suited for addressing rapid treatment titration after heart failure diagnosis. A very high-risk period for patients hospitalized for heart failure occurs when transitioning from a hospital to home. This transition period is exacerbated by challenges in drug titration.

The first 30-day window after heart-failure hospitalization is a transitional period with risk for need of readmission to a hospital. With this target in mind, some aspects herein can provide a wearable, disposable cardiac monitoring device, in particular, a continuous biochemical monitor for cardiovascular patients. As an illustrative example, three, disposable 2-week-worn monitoring devices (e.g., any one of the devices as described with regard to the preceding FIGURES) could be utilized, where the first monitoring device is applied during a patient's hospitalization or near time of first diagnosis, and would therefore allow monitoring for a sufficient period following, e.g., up to 24-42 days. Such a device facilitates large advances in medicine for decentralized care via remote monitoring, and can even assist in the hospital setting.

Aspects herein overcome the technical problem of cardiac patient monitoring, and provide a solution that goes well beyond the mere aggregation of disparate sensors. To the contrary, aspects herein facilitate the unification of multiple (previously distinct) sensing modalities, such that they can all be measured with a simplified electrode configuration, e.g., just a single gel-pad electrode outside the body and two electrodes inside the body. That is, what would normally be a five (or more) electrode system inside the body (working, working, working, counter, reference, etc.) is condensed into a two-electrode format for the monitoring device.

Moreover, the monitoring device herein can accomplish analysis more accurately than current artificial intelligence (AI) efforts that are attempting to non-invasively predict biomarkers such as Troponin and NT-proBNP, because the claimed measurement device directly measures the biomarker itself.

The monitoring device herein supports high-frequency patient monitoring during rapid-treatment titration. Applying a monitoring device described herein during rapid heart failure treatment titration can capture significant advances for heart failure management, such as to support improved patient outcomes; allows relatively more timely intervention if adverse events occur (e.g. hyperkalemia); provides a reduction in the complexity of health-care delivery through decentralized care; reduction of payer and patient costs; and provides a resolution of a very strong need for technology assistance for such patients.

### Example

An example monitoring device includes a group of sensors operatively configured for monitoring patients with heart issues, as described more fully herein. In this regard, the sensors can be tailored to the specific issues of the patient. For instance, for heart failure treatment, or for monitoring patients at risk of heart failure, the monitoring device can include a sensor for measuring NT-proBNP, potassium, tissue oxygen, or combinations thereof. Moreover, the monitoring device can optionally conduct at least one of electrocardiogram (ECG) measurements, heart rate and/or heart rate variability measurements, oxygen desaturation measurements, blood pressure measurements, or breathing rate measurements.

As another example, for AFIB diagnosis, the ECG and/or Breathing rate measurements can be effective, and for monitoring patients at risk of Afib, the monitoring device can include a sensor for measuring NT-proBNP, potassium, tissue oxygen, or combinations thereof. Moreover, the monitoring device can optionally conduct at least one of electrocardiogram (ECG) measurements, oxygen desaturation measurements, blood pressure measurements, or breathing rate measurements.

As noted more fully herein, accurate and timely measure of potassium can be effective at avoiding hypokalemia.

### Miscellaneous

Aspects herein combine chemicals, materials, sensors, electronics, microfluidics, algorithms, computing, software, systems, and other features or designs, combinations thereof, etc., that bring one or more sensing technologies into proximity with biofluid and analytes.

In this regard, novel approaches are provided herein, which simplify the integration of biochemical sensing with additional sensing modalities that enhance patient care or health and wellness.

In some implementations, the monitoring device, e.g., including in any of the configurations described more fully herein, can satisfy the needs of even a single subset of heart-disease patients. Moreover, in some implementations, the monitoring device could be used year-round in high-risk heart failure or AFib patients.

The terminology used herein is for the purpose of describing particular implementations only and is not intended to be limiting hereto. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description has been presented for purposes of illustration and description, but is not intended to be exhaustive or limited in the form disclosed. Many modifications and variations will be apparent to those of ordinary skill in the art without departing from the scope and spirit herein. Aspects were chosen and described in order to best explain the principles and the practical application of systems, processes and structures herein, and to enable others of ordinary skill in the art to understand various modifications as are suited to the particular use contemplated.

What is claimed is:

1. A wearable heart monitoring device, comprising:
a first electrode defining a sensor configured to measure at least one of b-type natriuretic peptide (BNP), N-terminal pro b-type natriuretic peptide (NT-proBNP) or Troponin; and
a second electrode configured to measure at least one parameter associated with heart disease or heart disease treatment of a patient wearing the heart monitoring device;
a housing coupled to the first electrode and the second electrode, the housing containing at least:
a potentiostat coupled to at least the first electrode;
a controller communicably coupled to the potentiostat;
memory coupled to the controller; and
a communication interface communicably coupled to the controller.

2. The wearable heart monitoring device of claim 1, wherein:
the first electrode is configured to measure at least one of b-type natriuretic peptide (BNP) or N-terminal pro b-type natriuretic peptide (NT-proBNP); and
the second electrode is configured to measure at least one parameter associated with heart disease or heart disease treatment of a patient by measuring at least one of potassium, creatinine or Troponin.

3. The wearable heart monitoring device of claim 2, wherein:
the second electrode is configured to measure potassium;
further comprising:
a third electrode configured to measure creatinine.

4. The wearable heart monitoring device of claim 1, wherein:
the first or second electrode is configured to measure oxygen concentration.

5. The wearable heart monitoring device of claim 1, wherein:
the first electrode or the second electrode functions as a standby sensor for detecting a heart attack by measuring Troponin.

6. The wearable heart monitoring device of claim 1 further comprising a gel electrode,
wherein:
with respect to the first electrode and/or the second electrode, the gel electrode is utilized for heart rate monitoring, and functions as a counter and/or reference for the first electrode and/or the second electrode.

7. The wearable heart monitoring device of claim 6, wherein the first electrode and the second electrode are provided on a carrier, and the carrier projects outwardly from the housing such that when applied to the patient, the gel pad electrode of the housing is operatively configured to be adherable outside the body, whereas the first electrode and the second electrode puncture the skin so as to reside inside the body.

8. The wearable heart monitoring device of claim 1 further comprising at least one additional sensor selected from:
an optical heart rate sensor;
an optical oxygen saturation sensor;
an accelerometer;
an electrocardiogramansor;
an optical pulse wave sensor configured for measuring blood pressure; or
an RF-measured thoracic-fluid index sensor.

9. The wearable heart monitoring device of claim 1 further comprising at least three sensors for simultaneous monitoring by the controller, of breathing rate, blood oxygen saturation (SpO₂), and tissue oxygenation so as to provide a measure of pulmonary-cardio-vascular performance.

10. The wearable heart monitoring device of claim 1, wherein:
the first electrode is initially not used;
the second electrode is configured as an electrocardiogram; and
the controller monitors the output of the electrocardiogram and issues an alert to have the first electrode inserted into the patient for monitoring thereof, if the monitored output of the electrocardiogram meets a predetermined criterion.

11. The wearable heart monitoring device of claim 1, wherein:
the controller is operatively configured to collect heart analytical data based upon measurements collected from the first electrode and the second electrode, and store the results in memory; and
the controller is further operatively configured to extract the heart analytical data from the memory and convey the data to a remote processing device.

12. A wearable heart monitoring device, comprising:
a housing having an adhesive thereon, the adhesive for securing the housing to a patient wearing the heart monitoring device;
a first electrode configured to measure a first cardiovascular parameter of a patient wearing the heart monitoring device;
a second electrode configured to measure a second cardiovascular parameter of the patient wearing the heart monitoring device; and
a third electrode functioning as a counter electrode for at least one of the first electrode and the second electrode;
wherein:
the first electrode and the second electrode are operationally configured as at least one needle electrode that is capable of puncturing through an epidermis of the patient;
the third electrode is operationally configured to attach to the outer skin of the patient;
the housing contains at least:
a potentiostat that couples to at least the first electrode;
a controller communicably coupled to the potentiostat;
memory coupled to the controller; and
a communication interface communicably coupled to the controller.

13. The wearable heart monitoring device of claim 12, wherein:
the first electrode is configured to measure the first cardiovascular parameter of the patient by measuring at least one of b-type natriuretic peptide (BNP) or N-terminal pro b-type natriuretic peptide (NT-proBNP).

14. The wearable heart monitoring device of claim 12, wherein:
the second electrode is configured to measure the second cardiovascular parameter of the patient by measuring at least one of potassium, creatinine or Troponin.

15. The wearable heart monitoring device of claim 12, wherein:
the first electrode or the second electrode is configured to measure oxygen concentration.

16. The wearable heart monitoring device of claim 12, wherein:
the third electrode further measures at least one of:
an electrocardiogrameasurement;
heart rate (HR);
heart rate variability (HRV);
HR+HRV;
Oxygen desaturation;
blood pressure;
breathing rate;
or combinations thereof.

17. The wearable heart monitoring device of claim 12, wherein:
the controller adjusts a voltage scanning window thus eliminating the need for a reference electrode.

18. A wearable heart monitoring device, comprising:
a housing for securing to a patient wearing the heart monitoring device;
a first electrode configured to measure a first cardiovascular parameter of a patient wearing the heart monitoring device; and
a second electrode configured to measure a second cardiovascular parameter of the patient wearing the heart monitoring device;
wherein:
the first electrode and the second electrode puncture through an epidermis of the patient; and
the housing contains at least:
a potentiostat that couples to at least the first electrode;
a controller communicably coupled to the potentiostat;
memory coupled to the controller; and
a communication interface communicably coupled to the controller.

19. The wearable heart monitoring device of claim 18, wherein:
the first electrode is configured to measure the first cardiovascular parameter of the patient by measuring at least one of b-type natriuretic peptide (BNP) or N-terminal pro b-type natriuretic peptide (NT-proBNP); and
the second electrode is configured to measure the second cardiovascular parameter of the patient by measuring at least one of potassium, creatinine or Troponin.

20. The wearable heart monitoring device of claim 18, wherein:
the controller adjusts a voltage scanning window thus eliminating the need for a reference electrode.
